(19) United States
(12) Patent Application Publication
(10) Pub. No.: US 2023/0326016 A1
(43) Pub. Date: Oct. 12, 2023

(54) ARTIFICIAL INTELLIGENCE FOR DETECTING A MEDICAL CONDITION USING FACIAL IMAGES

(71) Applicant: Kang ZHANG, Del Mar, CA (US)

(72) Inventor: Kang ZHANG, Del Mar, CA (US)

(21) Appl. No.: 18/118,869

(22) Filed: Mar. 8, 2023

Related U.S. Application Data
(63) Continuation of application No. PCT/US21/49483, filed on Sep. 8, 2021.
(60) Provisional application No. 63/075,802, filed on Sep. 8, 2020.

(51) Int. Cl. G06T 7/00; G06V 10/774; G06V 10/82; G06V 40/16; G06V 10/764; G06T 7/33; G06T 7/11; G06V 10/77; G16H 50/20; G16H 30/40

(52) U.S. Cl. CPC G06T 7/0012; G06V 10/774; G06V 10/82; G06V 40/171; G06V 10/764; G06T 7/337; G06T 7/11; G06V 10/7715; G16H 50/20; G16H 30/40; G06T 2207/20081; G06T 2207/20084; G06T 2207/30201; G06T 2207/30004; G06V 2201/03; G06T 2200/04; G06T 2207/20021; G06V 10/54

(57) ABSTRACT

Artificial intelligence for detecting a medical condition using facial images. In an embodiment, a convolutional neural network is applied to a facial image to identify facial landmarks, which are then used to align the facial image to a standard template. Next, the aligned facial image is projected into multi-views, and a second convolutional neural network is applied to the multi-views to extract global features. A facial-omics model is also applied to the aligned facial image to extract local features. A classification model is applied to the global features and the local features to predict one or more clinical parameters and/or medical conditions.

### BACKGROUND

The embodiments described herein are generally directed to artificial intelligence, and, more particularly, to artificial intelligence for detecting one or more medical conditions (e.g., metabolic or other disease) using facial images.

### SUMMARY

Systems, methods, and non-transitory computer-readable media are disclosed for detecting one or more clinical parameters and/or medical conditions (e.g., metabolic or other diseases) by applying artificial intelligence (AI) to facial images, such as two-dimensional (2D) and/or three-dimensional (3D) facial images.

In an embodiment, a method comprises using at least one hardware processor to: train an artificial intelligence to predict at least one clinical parameter or medical condition from facial images by training a first convolutional neural network to detect facial landmarks in each facial image, training a second convolutional neural network to predict one or more global features from each facial image, generating a facial-omics model to predict one or more local features from each facial image, and training a classification model to predict the at least one clinical parameter or medical condition based on the one or more global features and the one or more local features; and operating the trained artificial intelligence by, for each of a plurality of facial images, receiving the facial image, applying the first convolutional neural network to identify the plurality of facial landmarks in the facial image, aligning the facial image to a template based on the identified plurality of facial landmarks, applying the second convolutional neural network to the aligned facial image to predict the one or more global features, applying the facial-omics model to the aligned facial image to predict the one or more local features, and applying the classification model to the one or more global features and the one or more local features to generate a prediction of the at least one clinical parameter or medical condition for the facial image. Receiving the facial image may comprise receiving the facial image from a mobile device, which captured the facial image, over at least one network.

One or both of the first convolutional neural network and the second convolutional neural network may comprise a deep convolutional neural network. The second convolutional neural network may comprise a ResNet-50 in which a last global averaging layer is modified to produce an N-dimensional vector of global features, wherein N is greater than one hundred, such that the one or more global features comprise more than one-hundred global features.

Aligning the facial image to a template based on the identified plurality of facial landmarks may comprise computing a transformation that moves each of the identified plurality of facial landmarks in the facial image to a corresponding position of that facial landmark in the template.

Each received facial image may be a three-dimensional facial image, wherein applying the second convolutional neural network to the aligned facial image to predict the one or more global features comprises: projecting the aligned three-dimensional facial image into a plurality of two-dimensional directional views, wherein each of the plurality of two-dimensional directional views is a view of the three-dimensional facial image from a different angle than the other plurality of two-dimensional directional views; and applying the second convolutional neural network to the plurality of two-dimensional directional views to predict the one or more global features. The plurality of two-dimensional directional views may comprise a frontal view of a face in the three-dimensional facial image, one or more views of the face rotated in a leftward direction relative to the frontal view, one or more views of the face rotated in a rightward direction relative to the frontal view, one or more views of the face rotated in an upward direction relative to the frontal view, and one or more views of the face rotated in a downward direction relative to the frontal view. The one or more views of the face rotated in the leftward direction, the one or more views of the face rotated in the rightward direction, the one or more views of the face rotated in the upward direction, and the one or more views of the face rotated in the downward direction may all comprise a plurality of views at fixed intervals of rotation. Each plurality of views may comprise at least three views.

The facial image may be a three-dimensional facial image, wherein applying the facial-omics model to the aligned facial image to predict the one or more local features comprises: segmenting the three-dimensional facial image into a plurality of regions of interest; and applying the facial-omics model to the plurality of regions of interest to extract local features from each of the plurality of regions of interest. The plurality of regions of interest may be non-overlapping, wherein the plurality of regions of interest comprises a corner of right eye, right side of nose, upper right eye, right eye, lower right eye, chin, glabella, forehead, right cheek, philtrum, right temple, nose, mouth, corner of left eye, left side of nose, upper left eye, left eye, lower left eye, left cheek, and left temple. Segmenting the three-dimensional facial images into a plurality of regions of interest may comprise: representing the three-dimensional facial image as a face graph; and connecting subsets of the identified plurality of facial landmarks in the face graph into cycles representing the plurality of regions of interest. The facial-omics model may comprise principal component analysis. The local features may comprise one or both of one or more morphological features or one or more textural features. The local features may comprise a plurality of textural features, wherein the plurality of textural features comprises kurtosis, skewness, standard deviation, contrast, correlation, uniformity, directionality, homogeneity, coarseness, and directionality. The at least one clinical parameter or medical condition may comprise one or more of the following clinical parameters: age, weight, height, body mass index, smoking use, alcohol consumption, alanine aminotransferase, uric acid, hemoglobin concentrations, glutamyltransferase, hematocrit, and red blood cell volume. The at least one clinical parameter or medical condition may comprise one or more of the following medical conditions: obesity, diabetes, metabolic syndrome, hyperuricemia, nonalcoholic fatty liver disease, and anemia. The classification model may comprise a multilayer perceptron that outputs a vector of probabilities for a plurality of classifications representing the at least one clinical parameter or medical condition. The classification model may comprise a first model for predicting one or more clinical parameters other than age, a second model for predicting age, and a third model for predicting one or more medical conditions.

The method may be embodied in executable software modules of a processor-based system, such as a server, and/or in executable instructions stored in a non-transitory computer-readable medium.

### BRIEF DESCRIPTION OF THE DRAWINGS

The details of embodiments, both as to their structures and operations, may be gleaned in part by study of the accompanying drawings, in which like reference numerals refer to like parts, and in which:

FIG. 1 illustrates an example infrastructure, in which one or more of the processes described herein, may be implemented, according to an embodiment;

FIG. 2 illustrates an example processing system, by which one or more of the processes described herein, may be executed, according to an embodiment;

FIG. 3 illustrates an overall AI process, according to an embodiment;

FIG. 4 illustrates multi-views of a 3D facial image, according to an embodiment;

FIG. 5 illustrates regions of interest in a 3D facial image, according to an embodiment;

FIG. 6 illustrates the results of linear regression analysis on a plurality of clinical parameters, according to an embodiment;

FIG. 7 illustrates the performance of artificial intelligence in predicting a plurality of medical conditions, according to an embodiment;

FIGS. 8A-8D illustrate a metabolomics signatures analysis, according to an embodiment;

FIG. 9 illustrates the performance of artificial intelligence in predicting a plurality of medical conditions, according to an embodiment;

FIG. 10 illustrates the correlation of chronological age to predicted age and the impact of lifestyle on biological age, according to an embodiment;

FIG. 11 illustrates the performance of artificial intelligence in predicting a plurality of clinical parameters;

FIG. 12 illustrates the correlation between nicotinamide adenine dinucleotide (NAD+) and aging, according to an embodiment;

FIG. 13. Schematic illustration of the AI framework for 3D face representation and biometric/metabolic parameters analysis in an example of the present invention;

FIG. 14. The correlations between biometric and clinical parameters and 3D facial image features in an example of the present invention;

FIG. 15. Performance of the AI model on identification of metabolic diseases using 3D facial images in an example of the present invention;

FIG. 16. Metabolomics analysis linking metabolic disease and relating facial-omics in an example of the present invention;

FIG. 17. Mapping metabolites onto specific ROIs of 3D face in an example of the present invention;

FIG. 18. Performance of the AI model in a prospective point-of care pilot study using 3D images using a smartphone in an example of the present invention;

FIG. 19. Illustration of the 3D facial image preprocessing and standardization in an example of the present invention;

FIG. 20. Correlation of 3D facial image features with biometric and clinical parameters in an example of the present invention;

FIG. 21. Visualization of evidence for metabolic diseases prediction with ROI segments on a 3D face in an example of the present invention;

FIG. 22. Workflow chart for metabonomic analysis in an example of the present invention; and

FIG. 23. Metabolomics analysis of the metabolic disease and facial-omics in an example of the present invention.

### DETAILED DESCRIPTION

In an embodiment, systems, methods, and non-transitory computer-readable media are disclosed for detecting one or more clinical parameters and/or medical conditions (e.g., metabolic or other diseases) by applying artificial intelligence to facial images, such as 2D and 3D facial images. After reading this description, it will become apparent to one skilled in the art how to implement the invention in various alternative embodiments and alternative applications. However, although various embodiments of the present invention will be described herein, it is understood that these embodiments are presented by way of example and illustration only, and not limitation. As such, this detailed description of various embodiments should not be construed to limit the scope or breadth of the present invention as set forth in the appended claims.

### 1. INTRODUCTION

Due to the advancement of economic and health systems, the top cause of death has changed from infectious diseases to chronic diseases (Ackers and Malgor, 2018; Ricanati et al., 2011). Among chronic diseases, chronic metabolic diseases (CMD), including obesity, type 2 diabetes mellitus (T2DM), and non-alcoholic fatty liver disease (NAFLD), are the most prevalent and difficult to treat (Alberti et al., 2009). Diabetes and metabolic syndromes pose major challenges to health care. Diabetes is the most common disease, with over 382 million individuals estimated to be affected. Its prevalence has been increasing steadily in recent years and is expected to affect 629 million individuals by 2045. The U.S. Center for Disease Control (CDC) estimates that 12.2% of U.S. adults have T2DM. Even worse, 23.8% of these individuals are unaware of their condition, and therefore, do not seek effective therapy (Saklayen, 2018). According to the CDC, diabetes is the major risk factor for metabolic syndrome, characterized by obesity, glucose intolerance, and hyperlipidemia, which are leading risk factors for many common diseases, including cardiovascular disease, stroke, liver diseases, and kidney failure. Early diagnosis and treatment are crucial to reduce morbidities and mortality. The situation is especially serious as 7% of diabetic patients go undiagnosed.

Major lifestyle risk factors influence the state of an individual's health. Smoking and alcohol are the two major modifiable risk factors for metabolic syndrome. Liver diseases are another major disease group, heavily influenced by alcohol and smoking, and linked to diabetes and metabolic syndromes. Fatty liver disease is one of the most common liver diseases, affecting more than 100 million patients world-wide. Hence, a new system which can detect these diseases in the early phase or even predict the disease occurrence is highly desirable.

The human face is a multipartite trait composed of distinct features (e.g., eyes, nose, chin, mouth, and forehead). The size, shape, and composition of the human face are clearly distinct and show variations among individuals (Claes et al., 2018). Physicians can use facial appearance and expression to assess a patient's health status. For example, many diseases present a tell-tale sign, such as jaundice in hepatobiliary diseases, and mask-like expression in Parkinson's disease. In addition, many syndromes have recognizable facial features that are highly informative to physicians, such as telecanthus and cranial stenosis in down-syndrome patients (Roizen and Patterson, 2003). Moreover, multiple facial characteristics potentially act as cues to health judgments or actual health outcomes (Henderson et al., 2016). There is also evidence that individuals with facial adiposity have a higher risk of obesity and high blood pressure (Coetzee et al., 2009).

Recent advances in artificial intelligence have inspired innovations and applications in many healthcare areas (Kermany et al., 2018; Zhang et al., 2020). AI technologies have been successfully applied to facial analysis. In particular, artificial intelligence has achieved excellent performance in recognizing facial expressions, verifying a person's identity, and other related tasks, on the Labeled Faces in the Wild (LFW) dataset (Huang et al., 2008). Deep learning has been applied to the characterization of human faces and the identification of associations between facial morphology and personality traits, such as extraversion (Pound et al., 2007), achievement striving, deception (Haselhuhn and Wong, 2012), aggressiveness (Carre and McCormick, 2008), and risk-taking (Welker et al., 2015). Correlations between craniofacial characteristics and genetic disorders have been discovered both in clinical contexts (Ferry et al., 2014; Valentine et al., 2017) and in non-clinical contexts (Claes et al., 2014).

Although deep-learning technologies have been applied to facial recognition, they have not conventionally been used to evaluate health and disease status. The rich information encoded in the face has just begun to be explored for detection and diagnosis of biological traits and disease. Recent studies show that facial analysis technologies could be used to recognize genetic syndromes with a craniofacial phenotype for diagnoses of developmental syndromes (Gurovich et al., 2019). However, rich facial information has not been fully exploited for health monitoring and disease diagnoses for a number of reasons, including the prior unavailability of super-resolution three-dimensional (3D) cameras and powerful GPU-based computing capabilities. Recent studies have successfully demonstrated that the integration of artificial intelligence into both eye and childhood disease diagnostic systems can significantly improve clinical diagnostic efficiency and accuracy (Kermany et al., 2018), inspiring the hypothesis that the same benefits could be obtained in the realm of facial images and disease diagnoses.

While AI algorithms have advanced rapidly, their real-world application—particularly in facial-feature and disease-related applications—poses substantial challenges, including the lack of large training datasets, privacy issues surrounding data sharing, inadequate availability and distribution of algorithms, lack of data standardization, incompatibility of algorithms across multiple platforms, local regulatory requirements, and the like. Additionally, in low-resource settings, one of the key challenges is providing low-cost, fast, and accurate health status monitoring in a non-invasive way. In recognition of this limitation, deployment of AI-based technologies through mobile platforms has emerged as a growing area of investigation for digital point-of-care imaging systems. Smartphones are already built with the requisite hardware (e.g., structured light module) to capture the depth details necessary for 3D facial recognition by AI algorithms, such as FaceID, as well as object recognition by shopping apps. Compared to two-dimensional 2D facial images, 3D facial images have more depth and multi-view information, which can address the challenges of facial poses, uncontrolled ambient illumination, and aging and spoofing attacks (Taigman et al., 2014). With 3D facial images as one of the most prominent and accessible phenotypes of humans to convey information related to personal characteristics and health status, it is important to identify facial markers and their correlation with health indicators, and assess the risks of medical conditions, such as metabolic diseases.

One goal of the disclosed artificial intelligence was to develop a system capable of analyzing 3D facial images to detect common lifestyle risk factors and diseases. The general hypothesis was that a real-life photograph of an individual's face contains information on an individual's health and disease status that can be extracted using deep-learning techniques. As a proof of concept, diabetes, metabolic syndromes, and liver diseases were chosen for detection, since the identification and treatment of these conditions would provide a major improvement in healthcare. In an embodiment, the disclosed artificial intelligence was incorporated into a smartphone-based platform to provide a point-of-care system for screening of these common diseases. However, it should be understood that the disclosed artificial intelligence may be trained to detect any medical condition, including other diseases than those described, such as neurological diseases, psychological and/or psychiatric disorders, cancer, immunological diseases, dermatological diseases, congenital diseases, infectious diseases, and/or the like. In addition, the disclosed artificial intelligence may be incorporated into other platforms or systems than those described herein. Furthermore, while the artificial intelligence will be primarily described as being applied to 3D facial images, the artificial intelligence could alternatively be applied to 2D facial images, although it should be understood that 2D facial images generally contain less information than 3D facial images.

### 2. SYSTEMS

2.1. Infrastructure

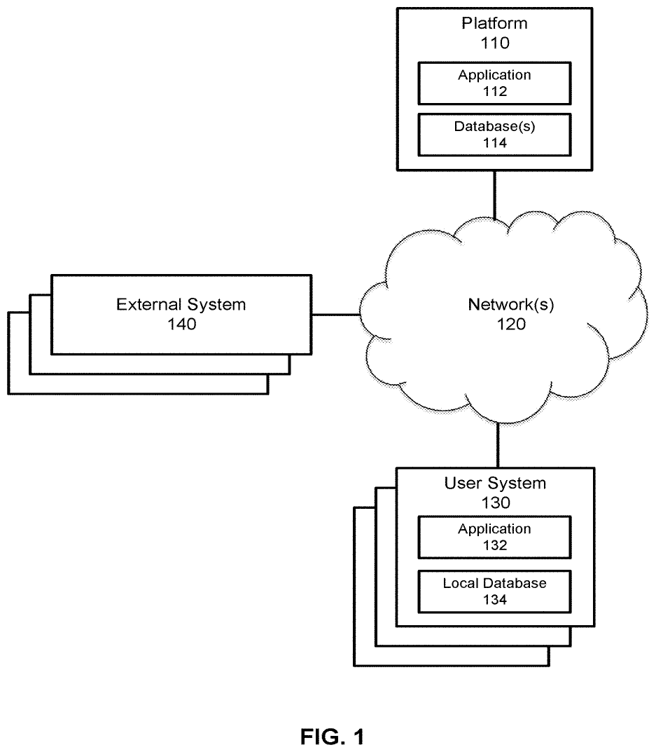

FIG. 1 illustrates an example infrastructure in which one or more of the disclosed processes (e.g., the disclosed artificial intelligence) may be implemented, according to an embodiment. The infrastructure may comprise a platform 110 (e.g., one or more servers) which hosts and/or executes one or more of the various functions, processes, methods, and/or software modules described herein, including the application which implements the disclosed artificial intelligence. Platform 110 may comprise dedicated servers, or may instead comprise cloud instances, which utilize shared resources of one or more servers. These servers or cloud instances may be collocated and/or geographically distributed. Platform 110 may also comprise or be communicatively connected to a server application 112 and/or one or more databases 114. In addition, platform 110 may be communicatively connected to one or more user systems 130 via one or more networks 120. Platform 110 may also be communicatively connected to one or more external systems 140 (e.g., other platforms, websites, etc.) via one or more networks 120.

Network(s) 120 may comprise the Internet, and platform 110 may communicate with user system(s) 130 through the Internet using standard transmission protocols, such as HyperText Transfer Protocol (HTTP), HTTP Secure (HTTPS), File Transfer Protocol (FTP), FTP Secure (FTPS), Secure Shell FTP (SFTP), and the like, as well as proprietary protocols. While platform 110 is illustrated as being connected to various systems through a single set of network(s) 120, it should be understood that platform 110 may be connected to the various systems via different sets of one or more networks. For example, platform 110 may be connected to a subset of user systems 130 and/or external systems 140 via the Internet, but may be connected to one or more other user systems 130 and/or external systems 140 via an intranet. Furthermore, while only a few user systems 130 and external systems 140, one server application 112, and one set of database(s) 114 are illustrated, it should be understood that the infrastructure may comprise any number of user systems, external systems, server applications, and databases.

User system(s) 130 may comprise any type or types of computing devices capable of wired and/or wireless communication, including without limitation, desktop computers, laptop computers, tablet computers, smartphones or other mobile phones, servers, game consoles, televisions, set-top boxes, electronic kiosks, point-of-sale terminals, point-of-care systems, and/or the like. In an embodiment, user system(s) 130 comprise ubiquitous mobile devices, such as smartphones, with high-resolution cameras capable of capturing 3D facial images.

Platform 110 may comprise web servers which host one or more websites and/or web services of the disclosed application. In embodiments in which a website is provided, the website may comprise a graphical user interface, including, for example, one or more screens (e.g., webpages) generated in HyperText Markup Language (HTML) or other language. Platform 110 transmits or serves one or more screens of the graphical user interface in response to requests from user system(s) 130. In some embodiments, these screens may be served in the form of a wizard, in which case two or more screens may be served in a sequential manner, and one or more of the sequential screens may depend on an interaction of the user or user system 130 with one or more preceding screens. The requests to platform 110 and the responses from platform 110, including the screens of the graphical user interface, may both be communicated through network(s) 120, which may include the Internet, using standard communication protocols (e.g., HTTP, HTTPS, etc.). These screens (e.g., webpages) may comprise a combination of content and elements, such as text, images, videos, animations, references (e.g., hyperlinks), frames, inputs (e.g., textboxes, text areas, checkboxes, radio buttons, drop-down menus, buttons, forms, etc.), scripts (e.g., JavaScript), and the like, including elements comprising or derived from data stored in one or more databases (e.g., database(s) 114) that are locally and/or remotely accessible to platform 110. Platform 110 may also respond to other requests from user system(s) 130.

Platform 110 may further comprise, be communicatively coupled with, or otherwise have access to one or more database(s) 114. For example, platform 110 may comprise one or more database servers which manage one or more databases 114. A user system 130 or server application 112 executing on platform 110 may submit data (e.g., user data, form data, etc.) to be stored in database(s) 114, and/or request access to data stored in database(s) 114. Any suitable database may be utilized, including without limitation MySQL™, Oracle™ IBM™, Microsoft SQL™, Access™, PostgreSQL™, and the like, including cloud-based databases and proprietary databases. Data may be sent to platform 110, for instance, using the well-known POST request supported by HTTP, via FTP, and/or the like. This data, as well as other requests, may be handled, for example, by server-side web technology, such as a servlet or other software module (e.g., comprised in server application 112), executed by platform 110.

In embodiments in which a web service is provided, platform 110 may receive requests from external system(s) 140, and provide responses in eXtensible Markup Language (XML), JavaScript Object Notation (JSON), and/or any other suitable or desired format. In such embodiments, platform 110 may provide an application programming interface (API) which defines the manner in which user system(s) 130 and/or external system(s) 140 may interact with the web service. Thus, user system(s) 130 and/or external system(s) 140 (which may themselves be servers), can define their own user interfaces, and rely on the web service to implement or otherwise provide the backend processes, methods, functionality, storage, and/or the like, described herein. For example, in such an embodiment, a client application 132 executing on one or more user system(s) 130 may interact with a server application 112 executing on platform 110 to execute one or more or a portion of one or more of the various functions, processes, methods, and/or software modules described herein. Client application 132 may be “thin,” in which case processing is primarily carried out server-side by server application 112 on platform 110. A basic example of a thin client application is a browser application, which simply requests, receives, and renders webpages at user system(s) 130, while the server application on platform 110 is responsible for generating the webpages and managing database functions. Alternatively, the client application may be “thick,” in which case processing is primarily carried out client-side by user system(s) 130. It should be understood that client application 132 may perform an amount of processing, relative to server application 112 on platform 110, at any point along this spectrum between “thin” and “thick,” depending on the design goals of the particular implementation. In any case, the application described herein, which may wholly reside on either platform 110 (e.g., in which case server application 112 performs all processing) or user system(s) 130 (e.g., in which case client application 132 performs all processing) or be distributed between platform 110 and user system(s) 130 (e.g., in which case server application 112 and client application 132 both perform processing), can comprise one or more executable software modules that implement one or more of the functions, processes, or methods of the application described herein.

2.2. Example Processing Device

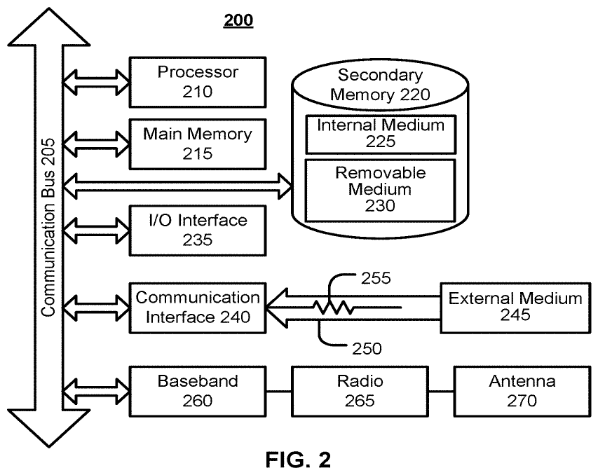

FIG. 2 is a block diagram illustrating an example wired or wireless system 200 that may be used in connection with various embodiments described herein. For example, system 200 may be used as or in conjunction with one or more of the functions, processes, or methods described herein (e.g., to store data for the disclosed artificial intelligence and/or execute the training and operation of the disclosed artificial intelligence), and may represent components of platform 110, user system(s) 130, external system(s) 140, and/or other processing devices described herein. System 200 can be a server or any conventional personal computer, or any other processor-enabled device that is capable of wired or wireless data communication. Other computer systems and/or architectures may be also used, as will be clear to those skilled in the art.

System 200 preferably includes one or more processors, such as processor 210. Additional processors may be provided, such as an auxiliary processor to manage input/output, an auxiliary processor to perform floating-point mathematical operations, a special-purpose microprocessor having an architecture suitable for fast execution of signal-processing algorithms (e.g., digital-signal processor), a slave processor subordinate to the main processing system (e.g., back-end processor), an additional microprocessor or controller for dual or multiple processor systems, and/or a coprocessor. Such auxiliary processors may be discrete processors or may be integrated with processor 210. Examples of processors which may be used with system 200 include, without limitation, the Pentium® processor, Core i7® processor, and Xeon® processor, all of which are available from Intel Corporation of Santa Clara, California.

Processor 210 is preferably connected to a communication bus 205. Communication bus 205 may include a data channel for facilitating information transfer between storage and other peripheral components of system 200. Furthermore, communication bus 205 may provide a set of signals used for communication with processor 210, including a data bus, address bus, and/or control bus (not shown). Communication bus 205 may comprise any standard or non-standard bus architecture such as, for example, bus architectures compliant with industry standard architecture (ISA), extended industry standard architecture (EISA), Micro Channel Architecture (MCA), peripheral component interconnect (PCI) local bus, standards promulgated by the Institute of Electrical and Electronics Engineers (IEEE) including IEEE 488 general-purpose interface bus (GPM), IEEE 696/S-100, and/or the like.

System 200 preferably includes a main memory 215 and may also include a secondary memory 220. Main memory 215 provides storage of instructions and data for programs executing on processor 210, such as one or more of the functions and/or modules discussed herein. It should be understood that programs stored in the memory and executed by processor 210 may be written and/or compiled according to any suitable language, including without limitation C/C++, Java, JavaScript, Perl, Visual Basic, .NET, and the like. Main memory 215 is typically semiconductor-based memory such as dynamic random access memory (DRAM) and/or static random access memory (SRAM). Other semiconductor-based memory types include, for example, synchronous dynamic random access memory (SDRAM), Rambus dynamic random access memory (RDRAM), ferroelectric random access memory (FRAM), and the like, including read only memory (ROM).

Secondary memory 220 may optionally include an internal medium 225 and/or a removable medium 230. Removable medium 230 is read from and/or written to in any well-known manner. Removable storage medium 230 may be, for example, a magnetic tape drive, a compact disc (CD) drive, a digital versatile disc (DVD) drive, other optical drive, a flash memory drive, and/or the like.

Secondary memory 220 is a non-transitory computer-readable medium having computer-executable code (e.g., disclosed software modules) and/or other data stored thereon. The computer software or data stored on secondary memory 220 is read into main memory 215 for execution by processor 210.

In alternative embodiments, secondary memory 220 may include other similar means for allowing computer programs or other data or instructions to be loaded into system 200. Such means may include, for example, a communication interface 240, which allows software and data to be transferred from external storage medium 245 to system 200. Examples of external storage medium 245 may include an external hard disk drive, an external optical drive, an external magneto-optical drive, and/or the like. Other examples of secondary memory 220 may include semiconductor-based memory, such as programmable read-only memory (PROM), erasable programmable read-only memory (EPROM), electrically erasable read-only memory (EEPROM), and flash memory (block-oriented memory similar to EEPROM).

As mentioned above, system 200 may include a communication interface 240. Communication interface 240 allows software and data to be transferred between system 200 and external devices (e.g. printers), networks, or other information sources. For example, computer software or executable code may be transferred to system 200 from a network server (e.g., platform 110) via communication interface 240. Examples of communication interface 240 include a built-in network adapter, network interface card (NIC), Personal Computer Memory Card International Association (PCMCIA) network card, card bus network adapter, wireless network adapter, Universal Serial Bus (USB) network adapter, modem, a wireless data card, a communications port, an infrared interface, an IEEE 1394 fire-wire, and any other device capable of interfacing system 200 with a network (e.g., network(s) 120) or another computing device. Communication interface 240 preferably implements industry-promulgated protocol standards, such as Ethernet IEEE 802 standards, Fiber Channel, digital subscriber line (DSL), asynchronous digital subscriber line (ADSL), frame relay, asynchronous transfer mode (ATM), integrated digital services network (ISDN), personal communications services (PCS), transmission control protocol/Internet protocol (TCP/IP), serial line Internet protocol/point to point protocol (SLIP/PPP), and so on, but may also implement customized or non-standard interface protocols as well.

Software and data transferred via communication interface 240 are generally in the form of electrical communication signals 255. These signals 255 may be provided to communication interface 240 via a communication channel 250. In an embodiment, communication channel 250 may be a wired or wireless network (e.g., network(s) 120), or any variety of other communication links. Communication channel 250 carries signals 255 and can be implemented using a variety of wired or wireless communication means including wire or cable, fiber optics, conventional phone line, cellular phone link, wireless data communication link, radio frequency (“RF”) link, or infrared link, just to name a few.

Computer-executable code (e.g., computer programs, such as the disclosed application, or software modules) is stored in main memory 215 and/or secondary memory 220. Computer programs can also be received via communication interface 240 and stored in main memory 215 and/or secondary memory 220. Such computer programs, when executed, enable system 200 to perform the various functions of the disclosed embodiments as described elsewhere herein.

In this description, the term “computer-readable medium” is used to refer to any non-transitory computer-readable storage media used to provide computer-executable code and/or other data to or within system 200. Examples of such media include main memory 215, secondary memory 220 (including internal memory 225, removable medium 230, and external storage medium 245), and any peripheral device communicatively coupled with communication interface 240 (including a network information server or other network device). These non-transitory computer-readable media are means for providing executable code, programming instructions, software, and/or other data to system 200.

In an embodiment that is implemented using software, the software may be stored on a computer-readable medium and loaded into system 200 by way of removable medium 230, I/O interface 235, or communication interface 240. In such an embodiment, the software is loaded into system 200 in the form of electrical communication signals 255. The software, when executed by processor 210, preferably causes processor 210 to perform one or more of the processes and functions described elsewhere herein.

In an embodiment, I/O interface 235 provides an interface between one or more components of system 200 and one or more input and/or output devices. Example input devices include, without limitation, sensors, keyboards, touch screens or other touch-sensitive devices, biometric sensing devices, computer mice, trackballs, pen-based pointing devices, and/or the like. Examples of output devices include, without limitation, other processing devices, cathode ray tubes (CRTs), plasma displays, light-emitting diode (LED) displays, liquid crystal displays (LCDs), printers, vacuum fluorescent displays (VFDs), surface-conduction electron-emitter displays (SEDs), field emission displays (FEDs), and/or the like. In some cases, an input and output device may be combined, such as in the case of a touch panel display (e.g., in a smartphone, tablet, or other mobile device).

System 200 may also include optional wireless communication components that facilitate wireless communication over a voice network and/or a data network (e.g., in the case of user system 130). The wireless communication components comprise an antenna system 270, a radio system 265, and a baseband system 260. In system 200, radio frequency (RF) signals are transmitted and received over the air by antenna system 270 under the management of radio system 265.

In an embodiment, antenna system 270 may comprise one or more antennae and one or more multiplexors (not shown) that perform a switching function to provide antenna system 270 with transmit and receive signal paths. In the receive path, received RF signals can be coupled from a multiplexor to a low noise amplifier (not shown) that amplifies the received RF signal and sends the amplified signal to radio system 265.

In an alternative embodiment, radio system 265 may comprise one or more radios that are configured to communicate over various frequencies. In an embodiment, radio system 265 may combine a demodulator (not shown) and modulator (not shown) in one integrated circuit (IC). The demodulator and modulator can also be separate components. In the incoming path, the demodulator strips away the RF carrier signal leaving a baseband receive audio signal, which is sent from radio system 265 to baseband system 260.

If the received signal contains audio information, then baseband system 260 decodes the signal and converts it to an analog signal. Then the signal is amplified and sent to a speaker. Baseband system 260 also receives analog audio signals from a microphone. These analog audio signals are converted to digital signals and encoded by baseband system 260. Baseband system 260 also encodes the digital signals for transmission and generates a baseband transmit audio signal that is routed to the modulator portion of radio system 265. The modulator mixes the baseband transmit audio signal with an RF carrier signal, generating an RF transmit signal that is routed to antenna system 270 and may pass through a power amplifier (not shown). The power amplifier amplifies the RF transmit signal and routes it to antenna system 270, where the signal is switched to the antenna port for transmission.

Baseband system 260 is also communicatively coupled with processor 210, which may be a central processing unit (CPU). Processor 210 has access to data storage areas 215 and 220. Processor 210 is preferably configured to execute instructions (i.e., computer programs, such as the disclosed application, or software modules) that can be stored in main memory 215 or secondary memory 220. Computer programs can also be received from baseband processor 260 and stored in main memory 210 or in secondary memory 220, or executed upon receipt. Such computer programs, when executed, enable system 200 to perform the various functions of the disclosed embodiments.

### 3. PROCESSES

Embodiments of processes for detecting one or more clinical parameters and/or medical conditions (e.g., metabolic or other diseases) by applying artificial intelligence to facial images (e.g., 2D and/or 3D facial images) will now be described in detail. It should be understood that the described processes may be embodied in one or more software modules that are executed by one or more hardware processors (e.g., processor 210), for example, as the application implementing the artificial intelligence discussed herein (e.g., server application 112, client application 132, and/or a distributed application comprising both server application 112 and client application 132), which may be executed wholly by processor(s) of platform 110, wholly by processor(s) of user system(s) 130, or may be distributed across platform 110 and user system(s) 130, such that some portions or modules of the application are executed by platform 110 and other portions or modules of the application are executed by user system(s) 130. The described processes may be implemented as instructions represented in source code, object code, and/or machine code. These instructions may be executed directly by hardware processor(s) 210, or alternatively, may be executed by a virtual machine operating between the object code and the hardware processors. In addition, the disclosed application may be built upon or interfaced with one or more existing systems.

Alternatively, the described processes may be implemented as a hardware component (e.g., general-purpose processor, integrated circuit (IC), application-specific integrated circuit (ASIC), digital signal processor (DSP), field-programmable gate array (FPGA) or other programmable logic device, discrete gate or transistor logic, etc.), combination of hardware components, or combination of hardware and software components. To clearly illustrate the interchangeability of hardware and software, various illustrative components, blocks, modules, circuits, and steps are described herein generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a component, block, module, circuit, or step is for ease of description. Specific functions or steps can be moved from one component, block, module, circuit, or step to another without departing from the invention.

Furthermore, while the processes, described herein, are illustrated with a certain arrangement and ordering of subprocesses, each process may be implemented with fewer, more, or different subprocesses and a different arrangement and/or ordering of subprocesses. For example, in some embodiments, one or more of the subprocesses may be omitted. In addition, it should be understood that any subprocess, which does not depend on the completion of another subprocess, may be executed before, after, or in parallel with that other independent subprocess, even if the subprocesses are described or illustrated in a particular order.

3.1. Overview

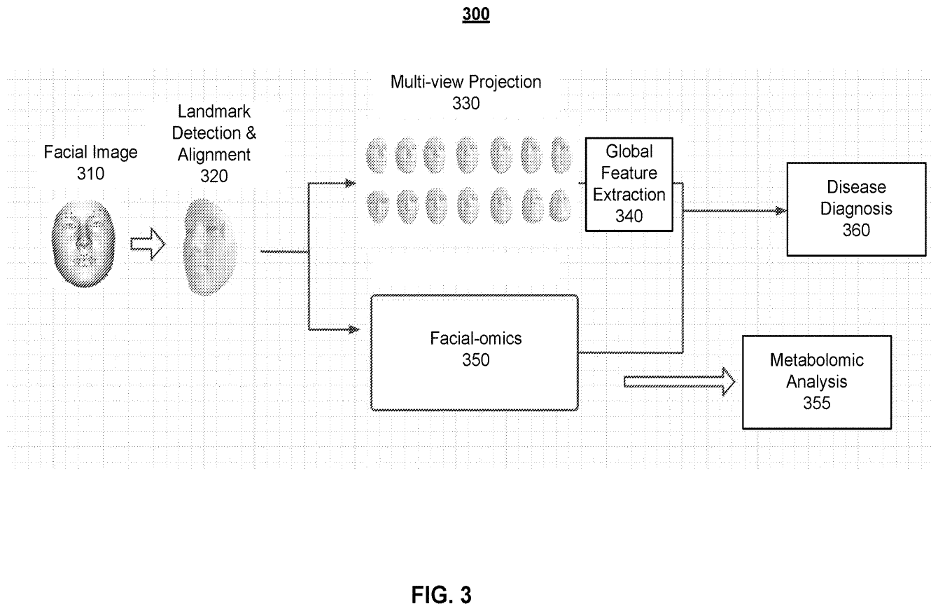

FIG. 3 illustrates a process 300 for detecting one or more clinical parameters and/or medical conditions by applying artificial intelligence to facial images, according to an embodiment. It should be understood that process 300 may be used for both training the artificial intelligence, as well as operating the artificial intelligence once it has been trained. It should also be understood that process 300 may be implemented by the disclosed application.

Initially, landmark detection and alignment subprocess 320 is applied to one or more facial images 310 to detect and align facial landmarks in received facial image(s) 310. In a preferred embodiment, each facial image 310 is a 3D image. However, in an alternative embodiment, each facial image 310 may be a 2D image. In an embodiment that uses 3D facial images 310, each 3D facial image 310 may be represented in the Wavefront.OBJ format. The Wavefront.OBJ format generates a dense 3D point cloud that represents the surface geometry of a human face from multiple 2D images having overlapping fields of view.

Multi-view projection subprocess 330 receives the aligned facial image(s) output by landmark detection and alignment subprocess 320, and generates a plurality of 2D projections or views of the facial surface. For example, in one particular implementation, multi-view projection 330 generates thirteen such projections. Each projection may represent a view of the facial surface from a different angle of rotation around the face.

Collectively, landmark detection and alignment subprocess 320 and multi-view projection subprocess 330 represent an automated pre-processing pipeline that detects 3D landmarks in facial images 310, transforms or normalizes the facial images 310 into a standard alignment based on the landmarks and a template, and rotates or projects the aligned facial images into a plurality of different views (i.e., multi-views). Three AI models for metabolic diseases and health status analysis may then be applied to the pre-processed facial images.

Global feature extraction subprocess 340 comprises a first AI model that receives the multi-views from multi-view projection subprocess 330. This first AI model may comprise a deep convolutional neural network (DCNN) that extracts the global features of the multi-views of an aligned facial image.

Facial-omics subprocess 350 comprises a second AI model that receives the aligned facial images from landmark detection and alignment subprocess 320. Facial-omics subprocess 350 performs high-throughput extraction of local feature information on quantitative descriptors from the aligned facial images. The extracted local feature information, referred to herein as “facial-omics,” can be used by metabolomic signatures analysis 355 and/or disease diagnosis subprocess 360. The underlying hypothesis of the facial-omics is that a facial image 310 can capture a full range of information on phenotypes of biological traits and medical conditions (e.g., diseases). In addition, metabolomic patterns, related to metabolic disease, may be reflected in facial images 310. Thus, assuming that imaging features are related to metabolic signatures, facial-omics can also be linked to the metabolomics of metabolic diseases.

Diagnosis subprocess 360 comprises a third AI model that receives the global features extracted by global feature extraction subprocess 340 and the local features or facial-omics extracted by facial-omics subprocess 350. This joint AI model combines the global features and local features to operate on a full facial representation and produce a diagnosis. The diagnosis may comprise predictions of one or more clinical parameters and/or medical conditions.

3.2. Landmark Detection and Alignment

An embodiment of landmark detection and alignment subprocess 320 will now be described in detail. At a high level, landmark detection and alignment subprocess 320 comprises two pre-processing steps: (1) detecting facial landmarks in a facial image 310; and (2) aligning the facial image 310 with a template based on the detected facial landmarks.

In an embodiment, the landmark detection comprises applying a deep convolutional neural network (DCNN) to detect a set of common facial landmarks. The set of facial landmarks should localize and represent salient regions of the face. For example, the method in Fagertun et al., 2014, may be used to detect a set of seventy-three such 3D facial landmarks.

In one particular implementation, the DCNN for landmark detection was initially trained to generate 2D heatmaps of landmark locations on 2D facial images (Paulsen et al., 2018). Then, 3D facial images were randomly projected into multiple views (e.g., one-hundred times) and input to the trained DCNN to generate 2D heatmaps. Finally, this information was propagated to a 3D space to estimate 3D landmarks. A least squares (LSQ) fit was combined with Random Sample Consensus (RANSAC) (Fisher and Bolles, 1980) selection to determine the accurate 3D positions of the 3D facial landmarks.

In an embodiment, once the positions of the facial landmarks are determined for a given facial image 310, those positions are aligned with a standard reference template for facial images (Claes et al., 2018). Specifically, a transformation matrix is computed that, when applied to 3D facial image 310, moves the detected 3D facial landmarks from its determined position to or near a position of the corresponding facial landmark in the reference template. Thus, similarity transformation matrices may be computed for each 3D facial image 310 using the reference template, to normalize all 3D facial images 310 according to a single common template. Using these transformations, spatially dense alignments may be established by matching points between the 3D facial images 310 and the reference template. However, it should be understood that the transformation matrices may provide rough, rather than exact, alignments.

3.3. Multi-View Projection

An embodiment of multi-view projection subprocess 330 will now be described in detail. It should be understood that multi-view projection subprocess 330 operates on each aligned facial image output by landmark detection and alignment subprocess 320.

Initially, a frontal view of the 3D facial surfaces is obtained by adjusting the horizontal direction according to the corners of the eyes in the aligned facial image, and adjusting the vertical direction according to a connection vector between the center of the corners of the eyes and the center of the corners of the mouth in the aligned facial image. Specifically, this adjustment may comprise rotating the 3D facial surfaces in the horizontal direction and/or vertical direction.

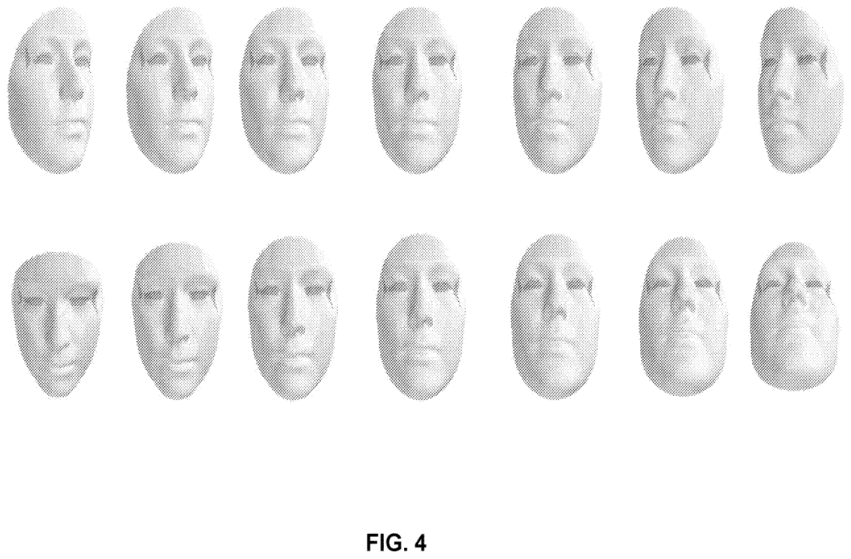

Next, the adjusted facial image is projected into a plurality of directional views. For example, the plurality of directional views may comprise views from N different directions, including a frontal view, a view for M intervals of 10 degrees of rotation up, a view for M intervals of 10 degrees of rotation down, a view for M intervals of 10 degrees of rotation left, and a view for M intervals of 10 degrees of rotation right. In the example multi-views illustrated in FIG. 4, M is equal three, such that N equals thirteen different views. However, it should be understood that different numbers of views and/or intervals, different intervals, and/or different sets of views may be used.

Finally, each 2D view or projection of the 3D facial surface may be cropped according to the positions of the facial landmarks detected by the landmark detection. For example, portions of each view representing background or irrelevant features (e.g., any region outside landmarks representing the edge of the face or the boundary of relevant features) may be removed from each projection.

3.4. Global Feature Extraction

An embodiment of global feature extraction subprocess 340 will now be described. Global feature extraction subprocess 340 operates on the multi-views of the aligned 3D facial images, output by multi-view projection subprocess 330. As described elsewhere herein, multi-view projection subprocess 330 may operate on an aligned 3D facial image to produce a plurality of 2D views (e.g., thirteen) of the face in the aligned 3D facial image. In an embodiment, global feature extraction subprocess 340 comprises applying a deep convolutional neural network (DCNN) to all of the multi-views to extract global features of the face, represented in facial image 310, from the multi-view projections. The DCNN has the property of shift invariance and space invariance. In an embodiment, each facial image that is input to global feature extraction subprocess 340 may be down-sampled or up-sampled into a common size (e.g., 512×512 pixels).

In one particular implementation of global feature extraction subprocess 340, a DCNN was pre-trained for global feature extraction using the IMDB-WIKI dataset (Rothe et al., 2015). The IMDB-WIKI dataset is a large-scale dataset of over 500,000 2D facial images with age and gender labels. ResNet-50 (He et al., 2016) was used as the backbone of the DCNN. ResNet-50 is a five-stage network with a convolution and four identity blocks. ResNet-50 utilizes skip connections to overcome the degradation problem of conventional deep-learning models. The last global averaging layer of the DCNN was modified to 512 nodes for 512-dimensional global feature extraction. However, it should be understood that the last global averaging layer may be modified to produce a different number of global features (e.g., dozens, one hundred, two hundred, three hundred, etc.). During pre-training using the IMDB-WIKI dataset, a fully connected layer with 101 nodes was appended to the DCNN. After pre-training, this fully connected layer was removed, and the DCNN, with the other structures and parameters retained, was used as the global feature extractor. In an embodiment, the output of the DCNN of global feature extraction subprocess 340 comprises a feature vector (e.g., a 512-dimensional feature vector).

3.5. Facial-Omics

An embodiment of facial-omics subprocess 350 will now be described in detail. It should be understood that facial-omics subprocess 350 operates on the aligned facial image output by landmark detection and alignment subprocess 320. Alternatively, facial-omics subprocess 350 could operate on each aligned and projected multi-view facial images output by multi-view projection subprocess 330.

In an embodiment, facial-omics subprocess 350 extracts quantitative descriptors (e.g., biometric and/or metabolic signatures) from the received facial images (e.g., 3D facial images) using regions of interest (ROIs). For example, the facial images may be segmented into one or more regions of interest. Then, local feature information, referred to herein as “facial-omics,” including morphological and/or textural features, may be extracted from each region of interest. A large number of facial-omics may be extracted. For example, in one particular implementation, 489 quantitative features were extracted as the facial-omics.

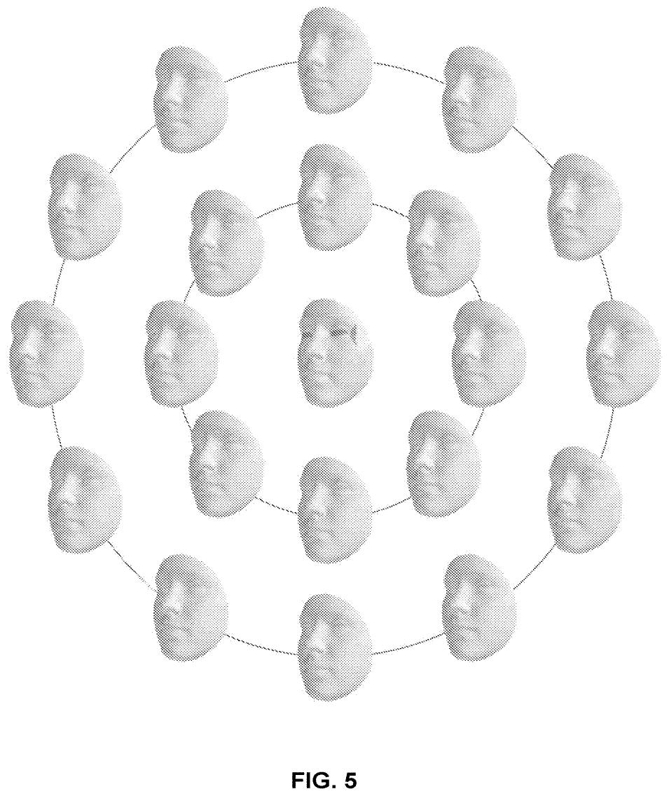

In an embodiment, the segmentation of the facial images into region(s) of interest is performed using a contour optimization approach (Clements and Zhang, 2006; Cohen, 2006) to automatically obtain one or more regions of interest. For example, in one particular implementation, illustrated in FIG. 5, each facial image was segmented into twenty non-overlapping regions of interest: corner of the right eye, right side of the nose, upper right eye, right eye, lower right eye, chin, glabella, forehead, right cheek, philtrum, right temple, nose, mouth, corner of the left eye, left side of the nose, upper left eye, left eye, lower left eye, left cheek, and left temple. However, in an alternative implementation, the plurality of regions of interest may overlap and/or the facial images may be segmented into a different number of regions of interest.

The landmarks and surfaces of a 3D facial image may be represented by a face graph, in which each region of interest is regarded as a sub-surface of the face graph or mesh, surrounded by a closed path (i.e., a contour or cycle). For each region of interest, a set of minimal paths, connecting pairs of facial landmarks, form a closed contour or cycle. Thus, the k-th region of interest can be represented as pₖ=[vₖ₁, vₖ₂, . . . , vₖₙ, vₖ₁], where vₖᵢ is the i-th landmark in pₖ. The path from vₖᵢ to vₖᵢ₊₁ was obtained using a shortest path algorithm on the face graph. Boundaries of the regions of interest in training datasets were manually refined to smooth the regions of interest and to ensure that the regions of interest collectively covered the whole 3D face. The table below depicts an example of ROI segmentation, showing all defined regions of interest in a 3D facial image with all corresponding landmarks:

| ROI | Landmarks Path | No. of Points |
|---|---|---|
| Eye corner right | [68, 70, 36, 17, 68] | 376 |
| Nose side right | [71, 72, 31, 39, 71] | 1,418 |
| Eye upper right | [36, 37, 38, 39, 21, 20, 19, 18, | 1,624 |
| | 17, 36] | |
| Eye right | [36, 37, 38, 39, 40, 41, 36] | 375 |
| Eye lower right | [70, 71, 39, 40, 41, 36, 70] | 912 |
| Chin | [5, 6, 7, 8, 9, 10, 11, 54, 55, 56, 57, | 7,695 |
| | 58, 59, 48, 5] | |
| Glabella | [39, 21, 20, 23, 22, 42, 28, 39] | 1,640 |
| Forehead | [19, 20, 23, 24, 79, 78, 19] | 2,680 |
| Cheek right | [0, 1, 2, 3, 4, 5, 48, 72, 71, 70, 0] | 6,607 |
| Philtrum | [72, 31, 32, 33, 34, 35, 77, 54, 53, | 1,414 |
| | 52, 51, 50, 49, 48, 72] | |
| Temple right | [0, 78, 19, 18, 17, 68, 70, 0] | 2,300 |
| Nose | [28, 39, 31, 32, 33, 34, 35, 42, 28] | 3,516 |
| Mouth | [48, 49, 50, 51, 52, 53, 54, 55, 56, | 1,217 |
| | 57, 58, 59, 48] | |
| Eye corner left | [73, 75, 45, 26, 73] | 525 |
| Nose side left | [76, 77, 35, 42, 76] | 1,393 |
| Eye upper left | [45, 44, 43, 42, 22, 23, 24, 25, 26, 45] | 1,706 |
| Eye left | [45, 44, 43, 42, 47, 46, 45] | 387 |
| Eye lower left | [75, 76, 42, 47, 46, 45, 75] | 916 |
| Cheek left | [16, 15, 14, 13, 12, 11, 54, 77, 76, | 6,744 |
| | 75, 16] | |
| Temple left | [16, 79, 24, 25, 26, 73, 75, 16] | 2,106 |

In an embodiment, after the facial images are segmented into the region(s) of interest, a principal component analysis (PCA) is applied to extract major features of morphological variations for each region of interest. PCA is an approach for reducing dimensionality and can eliminate some noisy and meaningless shape variations that result from various sources of error (Claes et al., 2018). The linear combination of principal components (PCs) from a given ROI segment can be extracted as the morphological features for that ROI segment. Given a defined region of interest R, a morphology vector M=(x₁, y₁, z₁, . . . , xₙ, yₙ, zₙ)∈ [symbol] ³ⁿ can be represented, in which n is the number of vertices containing x, y,z-coordinates. PCA can be performed for all morphology vectors of corresponding regions of interest from training data, so that morphological variations of R can be obtained using a linear combination of k reduced dimensions of principal components.

In an embodiment, textural features may be extracted for each region of interest in addition to or instead of morphological features. Textural features represent the spatially repetitive structure of surfaces, including local variations in scale, orientation, or other geometric attributes, which are important visual patterns in facial components (Kaesemodel Pontes et al., 2015). Initially, each facial image was converted to grayscale to reduce the inconsistency in colors.

Next, typical textural features were extracted for each region of interest. For example, in one particular implementation, ten textural features were extracted: kurtosis, skewness, standard deviation, contrast, correlation, uniformity, directionality, homogeneity, coarseness, and directionality. Among these ten textural features kurtosis, skewness, and standard deviation, are first-order statistical textural features. Kurtosis was extracted to describe the sharpness of the histogram, and skewness was defined as the degree of asymmetry around the mean value. Contrast, correlation, uniformity, directionality, and homogeneity are second-order statistical textural features (Lambin et al., 2012). A gray-level co-occurrence matrix (GLCM) may be used to analyze the spatial distribution of textural features in an image through different spatial positions and angles, so that the textural features are not influenced by the angle of rotation (Zhao et al., 2014). Contrast, uniformity, directionality, and homogeneity also belong to the set of visual textural features proposed by Tumura et al. (Tumura et al., 1978). Coarseness and directionality can also be quantified as facial-omics. Coarseness relates to the distances of dominant spatial variations of gray levels, i.e., implicitly to the size of the primitive elements (texels) forming the texture. The degree of directionality measures the frequency distribution of oriented local edges against their directional angles.

3.6. Disease Prediction

An embodiment of disease prediction subprocess 360 will now be described in detail. In an embodiment, disease prediction subprocess 360 applies an AI model to both the global facial features, output by global feature extraction subprocess 340, and the local features (facial-omics), output by facial-omics subprocess 350, to predict one or more—and preferably, a plurality—of clinical parameters that pertain to health status and medical conditions, such as metabolic diseases.

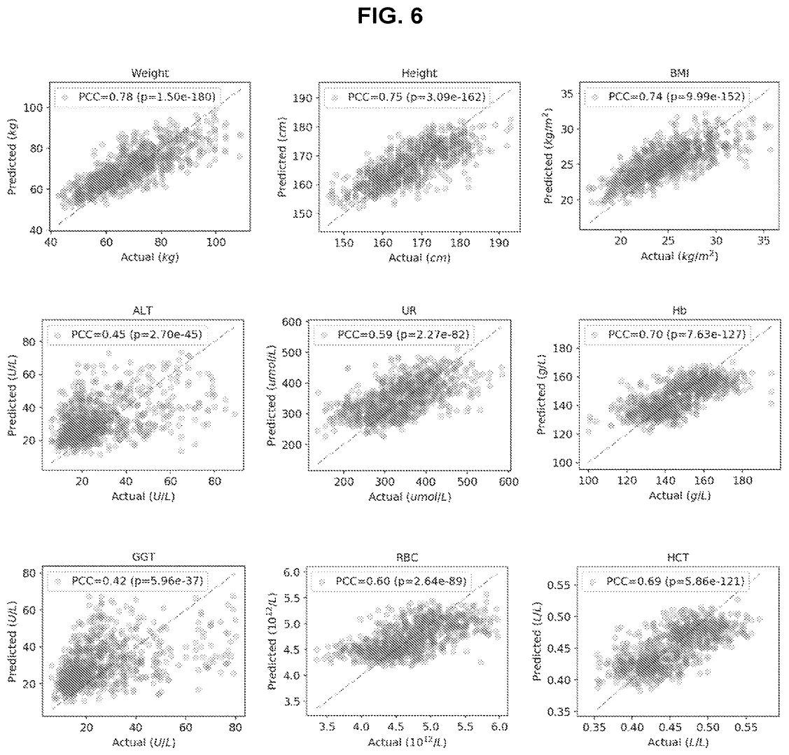

In one particular implementation, the AI model of disease prediction subprocess 360 was trained to simultaneously predict a plurality of clinical parameters, including height, weight, and body mass index (BMI). FIG. 6 shows the results of linear regression analysis on clinical parameters, that were predicted by the AI model of disease prediction subprocess 360, and the actual clinical parameters. As shown, weight, height, BMI, alanine aminotransferase (ALT), uric acid (UR), and hemoglobin concentrations (Hb) showed high positive correlations with facial features. Glutamyltransferase (GGT), hematocrit (Hct), and red blood cell volume (RBC) also showed relatively high positive correlations with facial features. These data demonstrate that facial images can be used to detect important clinical parameters that are highly relevant to metabolism and liver and blood functions, thereby laying the foundation for using facial features to predict relevant medical conditions.

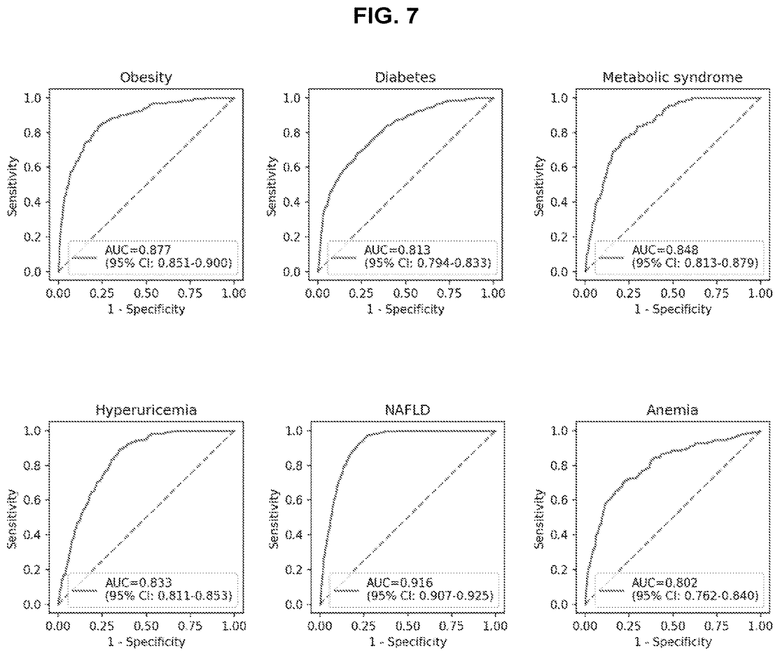

Since these data demonstrate strong correlations between facial images and clinical parameters that are highly relevant to metabolism and liver and blood functions, it was reasoned that the facial images could be used to directly predict a medical condition, such as a disease status. Therefore, in an embodiment, disease prediction subprocess 360 comprises a classification model to predict diseases using the clinical parameters, derived from facial images 310. In one particular implementation, binary classification models were trained to predict metabolic diseases, including obesity, diabetes, metabolic syndrome, hyperuricemia, NAFLD, and anemia. It should be understood that each binary classification model may classify a facial image as either normal or having the respective metabolic disease. Area under receiver operating characteristic (AUROC) curves were used to evaluate the performance of the model. As illustrated in FIG. 7, the model achieved an AUROC of 0.877 in predicting obesity, an AUROC of 0.813 in predicting diabetes, an AUROC of 0.848 in predicting metabolic syndrome, an AUROC of 0.833 in predicting hyperuricemia, an AUROC of 0.916 in predicting NAFLD, and an AUROC of 0.802 in predicting anemia. Thus, high accuracies were achieved across all disease categories. The performance of the model was externally validated using an independent dataset from a different geographic population in China.

Notably, metabolic syndrome is a complex mix of interrelated risk factors for cardiovascular disease (CVD) and diabetes (Grundy et al., 2005). Metabolic syndrome is defined by dyslipidemia (raised triglycerides and lowered high-density lipoprotein cholesterol), obesity, and diabetes (Alberti et al., 2009). Two out of four abnormal findings will quality a person for metabolic syndrome (Alberti and Zimmet, 1998). In the present description, hyperuricemia, NAFLD, anemia, and mental disorder are all considered metabolic diseases. Advantageously, disease prediction subprocess 360 was able to achieve a good predictive ability for metabolic syndrome defined in this manner.

Notably, evidence suggests that uric acid may play a role in the metabolic syndrome (Oh et al., 2009). Historically, the elevated level of uric acid observed in metabolic syndrome has been attributed to hyperinsulinemia. Hyperuricemia often precedes the development of obesity (Masuo et al., 2003) and diabetes (Dehghan et al., 2008). Advantageously, disease prediction subprocess 360 was able to achieve a good predictive ability for hyperuricemia.

In an embodiment, disease prediction subprocess 360 is implemented as a multilayer perceptron (MLP) that integrates the global features, output by global feature extraction subprocess 340, with the local features, output by facial-omics subprocess 350, to predict clinical parameters and/or disease classification. In one particular embodiment, for each subject, the model concatenated 512 global features with 489 local features. The MLP may be composed of two fully connected layers with rectified linear unit (ReLU) activation functions and a dropout rate of 0.2 to reduce overfitting. Each of the two layers is used for a different task. In an embodiment, the output of the MLP of disease prediction subprocess 360 is a set of one or more predicted classifications (e.g., as a vector of probabilities for a plurality of possible classifications, representing clinical parameters and/or medical conditions).

In an embodiment, disease prediction subprocess 360 comprises three separate models, with different last fully connected layers, for three tasks: a regression model for predicting clinical parameters (except age), a model (e.g., regression model) for predicting age (e.g., FaceAge), and a classification model for predicting metabolic diseases. The use of three separate models keeps the loss function on consistent scales. Mean-square error (MSE) loss was used as the objective function for regression of clinical parameters except for age, and the binary cross entropy (BCE) loss was used as the objective function for the multiple binary classification. Age prediction was separated from the regression model for predicting the other clinical parameters, because regression methods do not leverage a distribution's robustness in representing labels, such as ages, with ambiguity. However, age prediction could be implemented using an ordinal regression model (Pan et al., 2018).

In an embodiment, age prediction was treated as a distribution or classification problem, and the expected value over age probabilities, output by the softmax function, was used to predict age. Softmax weights may be used to calculate a weighted average age as the predicted age.

The objective function for age prediction may comprise three parts: focal loss (Lin et al., 2017), mean loss, and variance loss (Pan et al.). In particular, the focal loss was used to improve the prediction ability of the model for hard examples, by increasing their weight loss as follows:

$${L}_{f} = \frac{1}{N} {\sum }_{i = 1}^{N} - {( 1 - {p}_{{iy}_{i}} )}^{γ} log {p}_{{iy}_{i}}$$

wherein N is the batch size,
wherein P_{iy}_{ᵢ} denotes the probability that subject i belongs to the class j, and
wherein γ is the focusing parameter (e.g., γ=2).

The total loss is the weighted sum of focal loss, mean loss, and variance loss, which may be represented as follows:

$${L}_{f} = \frac{1}{N} {\sum }_{i = 1}^{N} [ - {( 1 - {p}_{{iy}_{i}} )}^{γ} log {p}_{{iy}_{i}} + {{α}_{1} ( {y}_{i} - {\sum }_{j = 1}^{K} j * {p}_{i , j} )}^{2} +$$

$${α}_{2} {\sum }_{k = 1}^{K} {p}_{{iy}_{i}} * {( j - {\sum }_{k = 1}^{K} k * {p}_{{iy}_{i}} )}^{2} ]$$

wherein experiments demonstrated that weights α₁=0.4 and α₂=0.05 worked well for age prediction (Pan et al., 2018).

In a particular implementation, all of the three separate models were implemented on Pytorch™ (Paszke et al., 2019), and optimized by the Adam algorithm (Kingma and Ba, 2014), with a learning rate of 0.001 and a weight decay of 10⁻⁴. The number of iterations was fifty with a batch size of thirty-two samples. The model training and evaluation was based on ten-fold cross validation. Thus, all samples were split into mutually exclusive sets for training and validation (90% of sample images) and testing (10% of sample images). This process was repeated ten times with different divisions of images, yielding ten mutually exclusive testing datasets that were collectively exhaustive. Augmentations were applied to the dataset to improve the generalization of the deep-learning models. These augmentations included oversampling (Masko and Hensman, 2015), brightness, contrast, saturation, and rotation.

3.7. Metabolomic Signatures Analysis

An embodiment of metabolomic signatures analysis 355 will now be described in detail. Human plasma samples were prepared according to the study in Contrepois et al., 2015. Samples were thawed on ice, prepared, and analyzed in a randomized order. Liquid chromatographic separation for processed plasma was achieved using a 100*2.1-mm ACQUITY™ UPLC BEH C18 1.7-μm column (Lot No. 0252350221) and an ACQUITY™ Ultra Performance LC, from Waters Corporation of Milford, Massachusetts. Mass spectrometry was performed using a SYNAPT™ G2 Quadrupole-Time of Flight system, from Waters Corporation. During analysis of the sample sequence, one quality control sample was run after every twenty injections. Data pre-treatment procedures, such as nonlinear retention time alignment, peak discrimination, filtering, alignment, matching, and identification, were performed using an XCMS package from Scripps Center for Metabolomics and Mass Spectrometry of La Jolla, California.

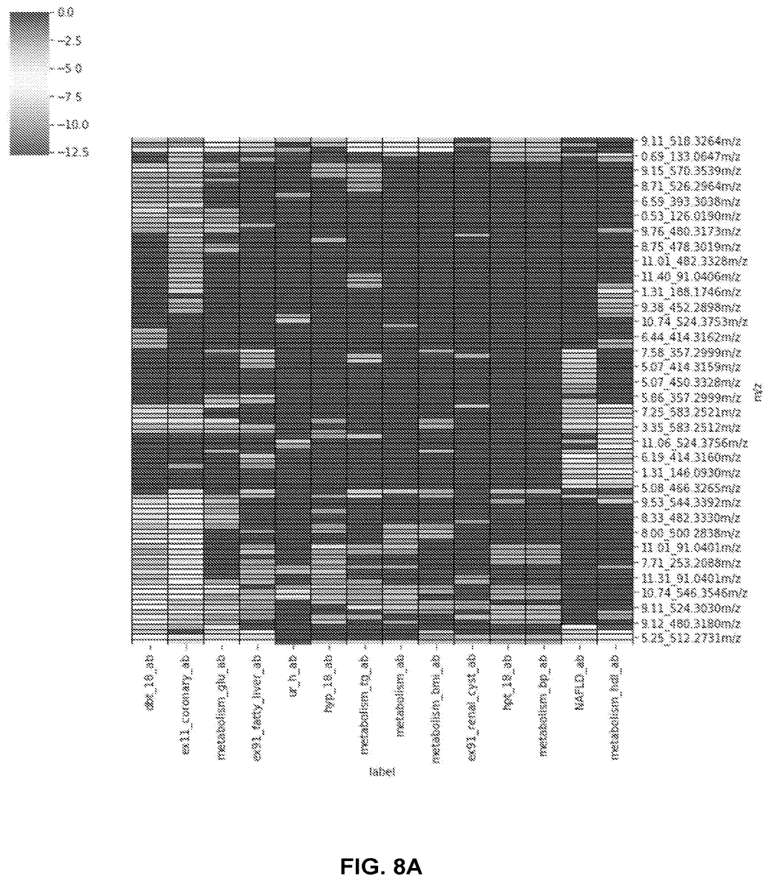

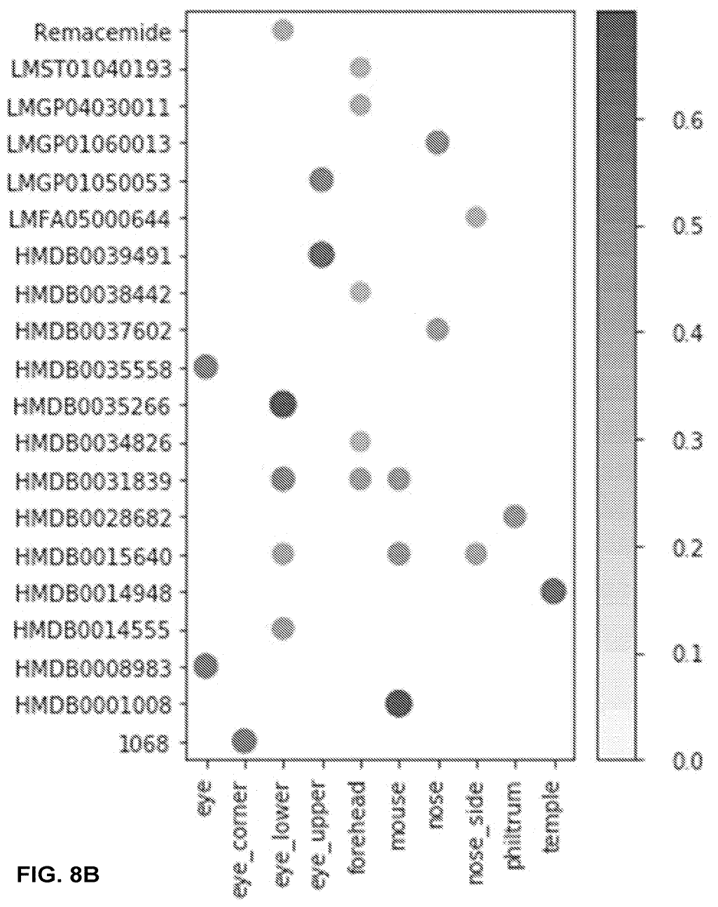

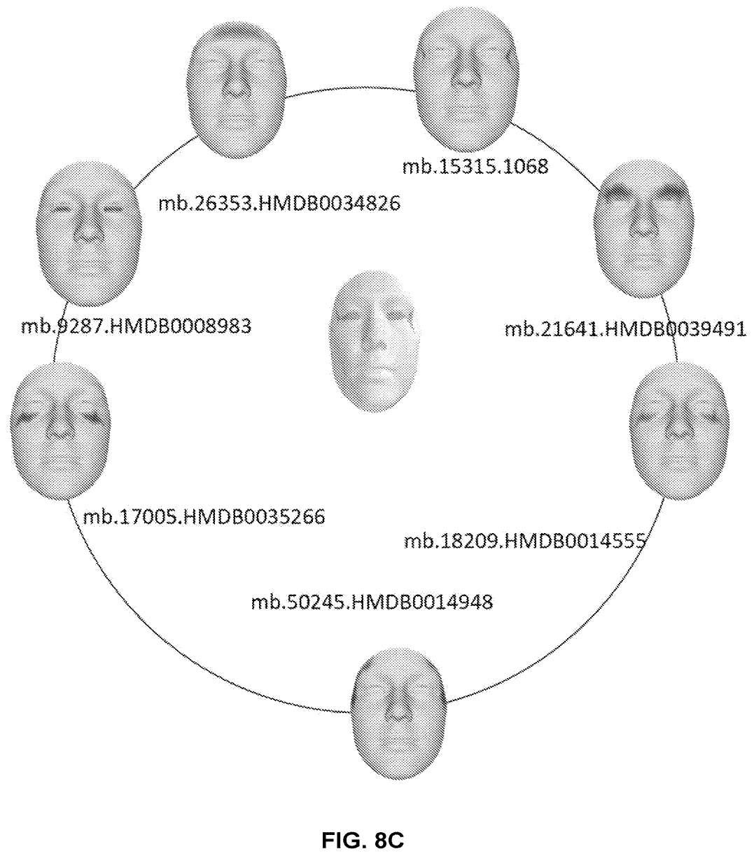

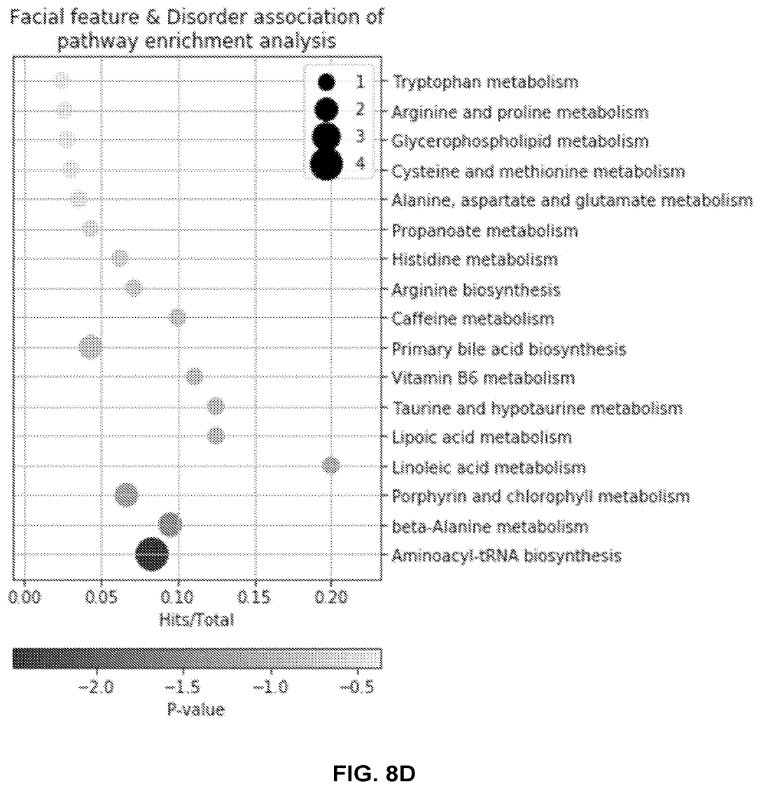

Patients' metabolic biomarkers, from health control and metabolic diseases, were identified using the Wilcoxon test and Student's t test. P values of multiple tests were adjusted using the false discovery rate (FDR). The cut-off for metabolic biomarkers was empirically set as 0.05 of adjusted p value. Pathway analyses of these metabolic biomarkers were analyzed in a MetaboAnalyst™ v.4 using the Kyoto Encyclopedia of Genes and Genomes (KEGG, Homo sapiens) pathway library (Chong et al., 2018). In addition, disease enrichment analyses were performed using the integrated enrichment analysis tool in MetaboAnalyst™. FIGS. 8A-8D illustrate the results of one particular implementation of the metabolomic signatures analysis 355.

### 4. TRAINING DATASET

In an embodiment, the training dataset for the disclosed artificial intelligence was constructed from 3D facial images in retrospective cohorts from the China Consortium of 3D Image Investigation (CC-3DF), which consists of the Yichang Central People's Hospital and the Han Chinese cohort at Tangshan, Heibei province. Institutional Review Board (IRB)/Ethics Committee approvals were obtained, and all patients signed a consent form. The 3D facial images were acquired using 3dMDface™ camera systems, produced by 3dMD LLC of Atlanta, Georgia, and represented in Wavefront.OBJ image files as point clouds and corresponding texture images. Applying standard facial image acquisition protocols, participants were asked to close their mouths and hold their faces with a neutral expression during capture of the digital facial stereophotogrammetry.

A total of 10,191 3D facial images were acquired from 7,072 subjects and used for training the disclosed artificial intelligence. The mean age of the subjects in the CC-3DF dataset was 46.93±13.67 years. A total of 4,921 subjects (i.e., 48.3%) from the CC-3DF dataset were male, and the mean body mass index (BMI) was 28.4±5.1 kg/m². Demographic information, lifestyle information (e.g., smoking, alcohol consumption, etc.), and clinical parameters (e.g., blood serum indicators, bloodcell-related indicators, etc.) were collected for each subject by routine physical examination. The clinical parameters may comprise, without limitation, age, height, weight, BMI, hemoglobin (Hb) concentrations, systolic blood pressure (SBP), diastolic blood pressure (DBP), glutamyltransferase (GGT), creatinine (Cr), hematocrit (Hct), red blood cell (RBC) and liver function indicators (e.g., alanine aminotransferase (ALT) and uric acid (UR)), aspartate transaminase (AST), and/or the like. Metabolic disease labels were also collected. The cohort characteristics and listing of targeted proteins (immune, cardiovascular, and metabolic) and reported plasma and cellular analytes are depicted in the table below:

| Characteristic | No. of subjects |
|---|---|
| Hyperuricemia | 309 |
| Diabetes | 678 |
| Metabolic Syndrome | 340 |
| Obesity | 268 |
| Non-alcoholic Fatty | 1,269 |
| Liver Disease (NAFLD) | |
| Anemia | 264 |
| Smoking | 967 |
| Drinking | 1,406 |
| Mean UR (umol/L) | 340.64 ± 88.09 |
| Mean ALT (U/L) | 30.05 ± 23.58 |
| Mean V5 (U/L) | 29.82 ± 26.60 |
| Mean AST (U/L) | 25.45 ± 13.06 |
| Mean TG (U/L) | 1.83 ± 1.50 |
| Mean Hb (g/L) | 145.30 ± 16.82 |
| Mean HcT (L/L) | 0.4473 ± 0.0474 |
| Mean RBC (10¹²/L) | 4.70 0.49 |

It should be understood that a larger training dataset may produce a more accurate AI model. Training with significantly larger datasets and with more clinical metadata may provide further evidence that 3D facial images can augment or replace one or more of the biomarkers used to detect metabolic disease and its associated risk factors.

### 5. PILOT STUDY

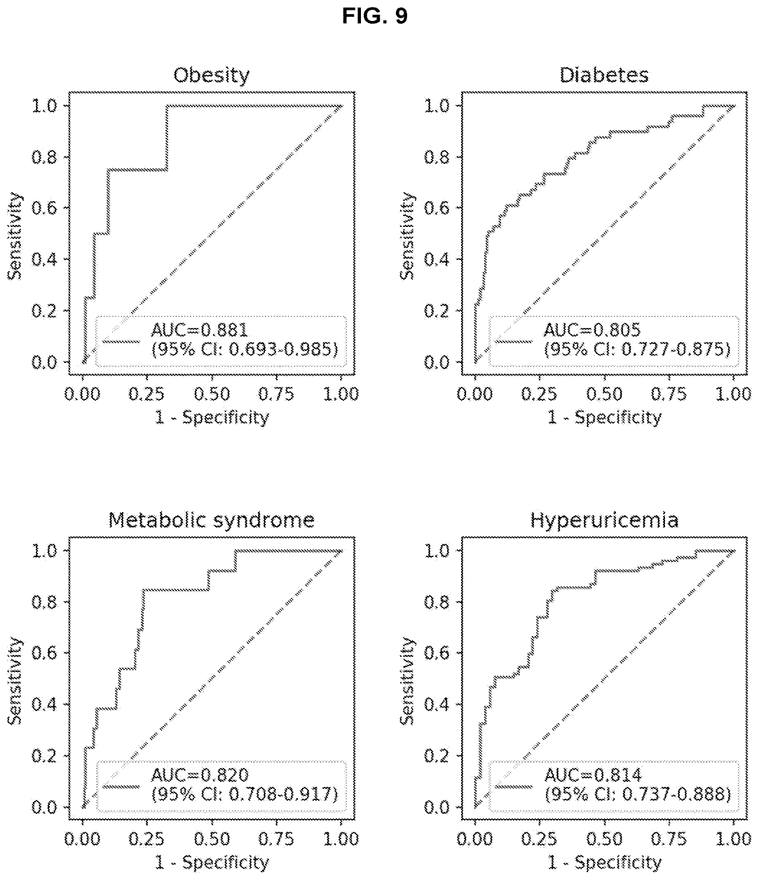

Prospective pilot studies were conducted using 3D facial images, generated from a smartphone, to test the performance of the disclosed artificial intelligence. In these point-of-care studies of 3D facial images and related clinical parameters, the 3D facial images were captured using a structured light module of a smartphone and represented in the Wavefront.OBJ format. In one study, a total of 350 patients were included in an external clinical validation dataset, with a mean age of 56.9±5.1 kg/m², 58% male, and a mean BMI of 28.4±5.1. FIG. 9 illustrates the performance of the artificial intelligence in predicting obesity, diabetes, metabolic syndrome, and hyperuricemia, according to an embodiment. Characteristics of the external clinical validation dataset are depicted in the table below:

| Characteristic | No. of subjects |
|---|---|
| Obesity | 4 (n = 230) |
| Diabetes | 49 (n = 208) |
| Metabolic Syndrome | 13 (n = 206) |
| Hyperuricemia | 77 (n = 132) |

### 6. ASSOCIATIONS WITH AGE, GENDER, AND LIFESTYLE FACTORS

The correlations between actual clinical parameters and clinical parameters, predicted by the disclosed artificial intelligence from 3D facial images, were investigated. The models of disease diagnosis module 360 were trained using MSE loss to regress clinical parameters, including height, weight, BMI, ALT, UR, SBP, DBP, GGT, Cr, Hct, and RBC. Pearson's correlation tests were performed between actual values and predicted values. Resultant correlations were regarded as significant when their P values were less than 0.001.

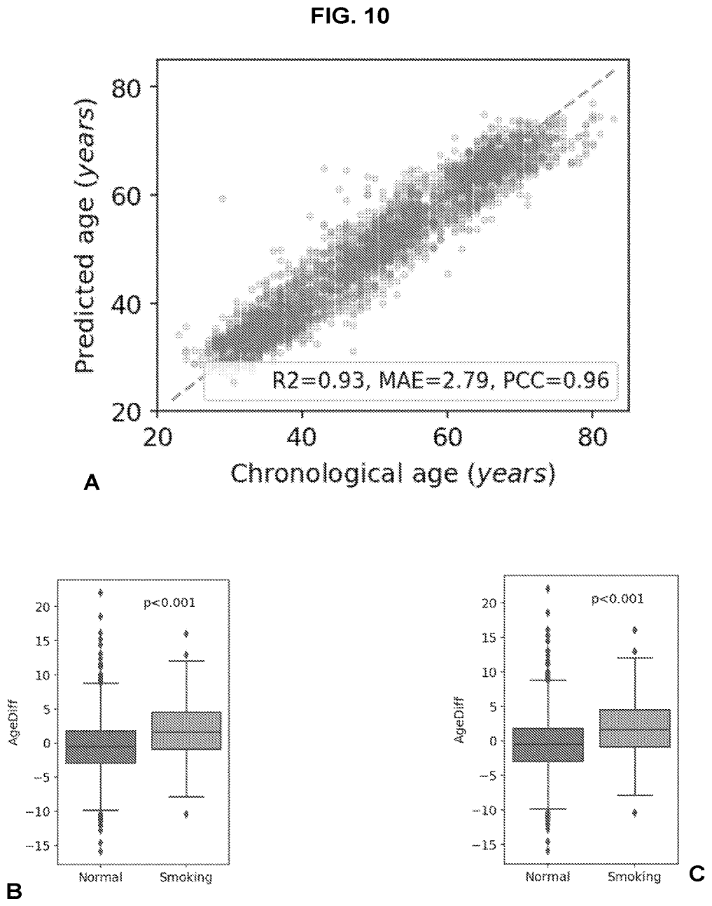

Since the effects of aging are highly visible in a human face and old age is a common risk factor for metabolic diseases, in a particular implementation, the disclosed artificial intelligence was trained to predict chronological age. This model is referred to herein as “FaceAge,” and its output is referred to herein as “face age.” It was assumed that face age could be a potential biomarker of biological age (BA) (Jia et al., 2017). If rich aging-related information could be visualized non-invasively from the face, FaceAge could be used to help quantify the individual differences in senescence of a specific system or organ. A linear regression analysis was implemented to identify the association of the predicted face age to the chronological age. The FaceAge model's predictions of face age had a strong linear relationship to the chronological age, with a Pearson's correlation coefficient (PCC) of 96% and a mean absolute error (MAE) of 2.7 years. This is illustrated in FIG. 10, which depicts the following graphs: (A) the correlation of chronological age and the face age predicted using 3D facial images; (B) the increased biological age of smokers compared to non-smokers; and (C) the increased biological age of alcohol users compared to non-alcohol users. The coefficient of determination (R2) value in graph (A) is a measure of the proportion of variation in the dependent variable that can be attributed to the independent variable. The R2 of 0.92, achieved by the FaceAge model, demonstrates that the FaceAge model can fit data and predict chronological age with high precision. The age difference (AgeDiff) analysis in graphs (B) and (C) measures the difference of predicted face age and chronological age for respective lifestyle groups. Each box plot gives a median, upper quartile, and lower quartile by the box and the upper adjacent value and lower adjacent value by the whiskers. Two-tailed Wilcoxon rank-sum tests were used to determine significance.

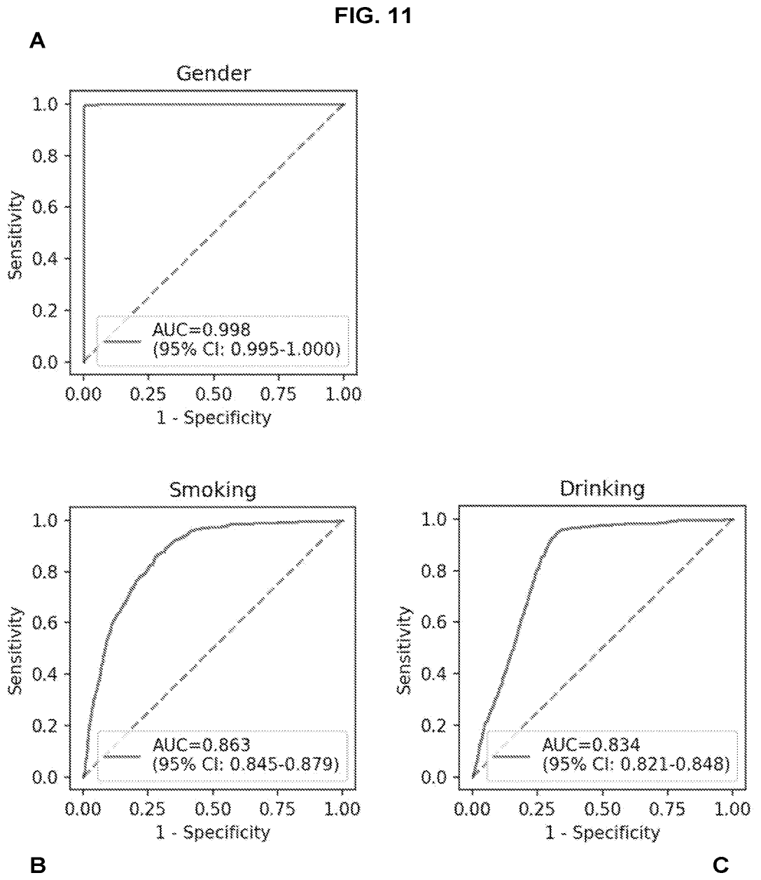

In a particular implementation, the artificial intelligence was also trained to predict gender and lifestyle factors, such as smoking and alcohol use, from facial images. The AUROC curves were calculated to evaluate the AI model's ability to distinguish male versus female genders and predict smoking and alcohol use. As illustrated in FIG. 11, the artificial intelligence achieved: (A) an AUROC of 0.996 in predicting gender based on facial images; (B) an AUROC of 0.863 in predicting current smoking status; and (C) an AUROC of 0.834 in predicting current alcohol use.

Since lifestyle factors can impact the aging process, smoking and alcohol consumption were investigated for their ability to modify the predicted face age. Specifically, the artificial intelligence was trained using a dataset comprising facial images of 3,584 subjects without a habit of smoking and alcohol consumption, using ten-fold cross validation. Then, the trained artificial intelligence was applied to predict face age for 1,244 subjects with a habit of smoking and 1,849 subjects with a habit of alcohol consumption. The cross validation was performed at the patient level, guided by a patient-specific identifier, to ensure that all facial images from the same patient were allocated to, at most, one subset per validation. The two-side P value was computed by t test with AgeDiff. The AgeDiff was defined by the difference between the predicted face age and the chronological age (e.g., predicted face age minus chronological age). It was found that smoking can positively affect the predicted face age in a wide age range (P<0.001). Similarly, alcohol consumption can accelerate predicted face age (P<0.001).

To study the underlying molecular mechanism on aging, metabolism and energy pathways were studied, since these have been implicated in the aging process. For example, the correlation between biological age and nicotinamide adenine dinucleotide (NAD+) for subjects was studied. Levels of NAD+ in the subjects' blood were measured using a cycling assay and high-performance liquid chromatography (HPLC) assay, according to standard protocols. Four cycling-assay, 40 μL blood samples were extracted with 0.5M HClO₄, neutralized by 3M KOH on ice and 125 mM Gly-Gly buffer (pH 7.4) on ice, then centrifuged at 10,000×g for fifteen minutes. Supernatants were mixed with a reaction medium containing 0.1 mM WST-8, 0.9 mM phenazinemethosulfate (PMS), 13 units/ml alcohol dehydrogenase, 100 mM nicotinamide, 5.7% ethanol in 61 mM Gly-Gly buffer (pH 7.4). Samples were mixed in a 96-well plate at room temperature. The A450 nm was determined immediately and after 20-30 minutes, and the results were calibrated with NAD+ standards. Total NAD+ was quantified using a plate reader. Because of variations in sample processing, samples were processed in parallel. Normalized values from each experiment were used to obtain average values. For the HPLC assay, the supernatant was loaded into a Hypersil Gold aQ C18 column, 5 μm particle size (250×4.6 mm, Thermo Fisher Scient). The HPLC was run at a flow rate of 1 mL/min. with 100% buffer A (0.05 M phosphate buffer) from 0-5 minutes, a linear gradient to 95% buffer A/5% buffer B (100% methanol) from 5-6 minutes, 95% buffer A/5% buffer B from 6-11 minutes, a linear gradient to 85% buffer A/15% buffer B from 11-13 minutes, 85% buffer A/15% buffer B from 13-23 minutes, a linear gradient to 100% buffer A from 23-24 minutes, and 100% buffer A from 24-30 minutes. The pressure through the HLPC system was carefully monitored during the measurement. NAD+ is monitored by absorbance at 261 nm. The peak for NAD+ is eluted as a sharp peak at 17 minutes and completely separable from peaks for other metabolites. NAD+ levels were quantified based on the peak area compared to a standard curve.

After predicting the age for healthy subjects, a bootstrap was applied (Efron, 1992) to determine whether there was a statistical significance between face-age-NAD distribution and chronological-age-NAD distribution. Resampling was performed one thousand times, and the PCC was computed each time. Finally, dependent t tests for paired samples were used for statistical analysis.

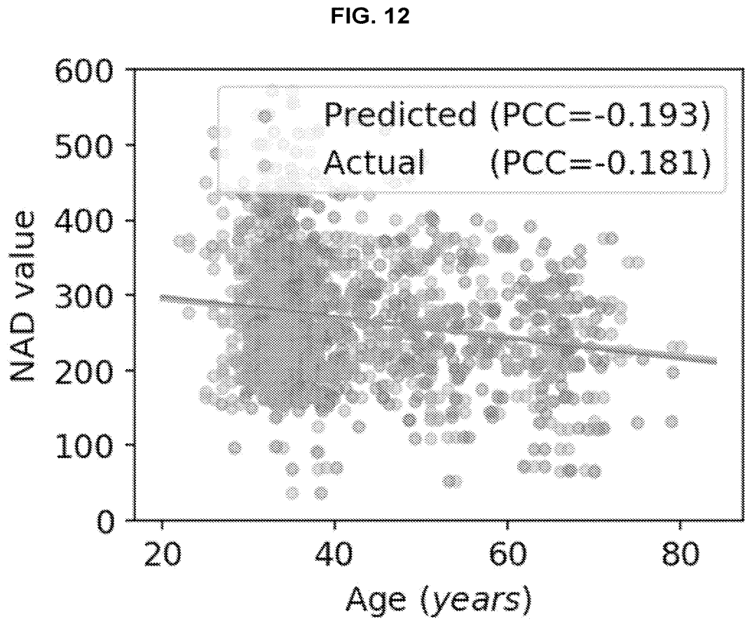

As illustrated in FIG. 12, a significant correlation was found between the gradual decline in NAD+ and aging. NAD+ is an essential electron transporter in mitochondrial respiration and oxidative phosphorylation. NAD+ is also the sole substrate for the nuclear repair enzyme, poly(ADP-ribose) polymerase (PARP) and the sirtuin family of NAD-dependent histone deacetylases. Depletion of NAD+ levels is strongly correlated with aging in both rodents and humans, and repletion retards the aging process (Verdin, 2015; Zhu et al., 2015). Thus, the correlations of NAD+ levels with predicted age and chronological age were investigated. A negative correlation was found between NAD+ levels and predicted age, which was more significant than with chronological age. The results indicate that biological age, derived from facial imaging, can be used as an effective biomarker of aging.

### 7. POINT-OF-CARE SYSTEM

There is potentially broad appeal for an AI-based medical diagnosis system that is based on non-invasive facial imaging. Thus, in an embodiment, the disclosed application, implementing process 300, representing the disclosed artificial intelligence, supports a point-of-care system that diagnoses common diseases using facial images 310 acquired by a smartphone (e.g., the camera of an Apple iPhone™ 10), or other ubiquitous mobile device, as user system 130. The digital cameras on current mobile devices generally have sufficient resolution to obtain a 3D model with acceptable precision for the disclosed artificial intelligence.

In one particular implementation, client application 132, executing on a smartphone, provides a graphical user interface that guides a user through acquisition of a 3D facial image 310 using the camera of the smartphone. Client application 132 then uploads the captured 3D facial image 310 to server application 112 (e.g., a cloud service) on platform 110. Platform 110 is preferably compliant with the Health Insurance Portability and Accountability Act (HIPAA). Server application 112 implements process 300 to autonomously make a diagnosis based on the uploaded 3D facial image 310.

Even when the artificial intelligence was trained and tested using 3D facial images 310 captured using standard 3D cameras, in operation, the artificial intelligence achieved comparable overall average diagnostic accuracies using 3D facial images 310 that were captured using smartphone cameras. For example, the correlations between chronological and predicted face age were high (R2=0.84, PCC=0.92). In addition, the artificial intelligence achieved an AUROC of 0.881 for predicting BMI, an AUROC of 0.805 for predicting diabetes, and an AUROC of 0.801 for predicting metabolic syndrome. These results demonstrate that the disclosed artificial intelligence has real-world viability for facial-based diagnosis via personal mobile devices.

### 8. QUANTIFICATION AND STATISTICAL ANALYSIS

The performance of the disclosed artificial intelligence for age prediction was evaluated using three metrics: Mean Absolute Error (MAE), R-square (R2), and Pearson Correlation Coefficient (PCC). MAE is the measure of errors between predicted age and chronological age. R2 is a statistical measure that represents the proportion of the variance for predicted face age, explained by chronological age, in the deep-learning model. PCC was used to measure the correlation between two variables. PCC has a value between +1 and −1, where +1 and −1 represent total complete linear correlation and 0 represents no linear correlation. The significance of correlation between two distributions was computed using a bootstrapping approach (Efron, 1992) with a resampling of one-thousand times.

Receiver operating characteristics (ROC) and AUROC were used to assess model performance for each classification task. The ROC curves were plotted by using the true positive rate (sensitivity) versus the false-positive rate (1−specificity). Sensitivity, specificity, and accuracy were determined by selected thresholds. A weighted error was used to evaluate models and experts, to reflect clinical performance. The Python™ scikit-learn library was used for data analysis, including measurements of sensitivity, specificity, and accuracy. The Python™ matplotlib and seaborn libraries were used to plot graphs.

### 9. EXAMPLE USAGE

Risk stratification is central to screening and managing patients at risk for metabolic syndromes, which are a leading cause of death world-wide. Although there are available metabolic syndrome risk calculators, such as lipid-based, BMI-based, and cholesterol-based composite score systems, many efforts have been made to improve risk predictions and population-based screening. The current standard of care for the screening of the risk of metabolic syndrome requires a variety of variables derived from the patient's history and blood samples, such as age, gender, smoking status, blood pressure, BMI, glucose, and cholesterol levels (Goff et al., 2014). Most metabolic syndrome risk calculators use some combination of these parameters to identify patients at risk of significant vascular-related morbidity and mortality (Cooney et al., 2009; Dudina et al., 2011; Goff et al., 2014; Poplin et al., 2018). However, some of these parameters may be difficult to obtain and unavailable for large-scale population screening. Therefore, the disclosed artificial intelligence can advantageously predict the risk of metabolic syndrome from facial images, which can be obtained quickly, cheaply, and non-invasively, for example, at the patient's home (e.g., using the patient's smartphone).

Although deep learning has been successfully applied to facial recognition, including person identification, its application in health care and disease detection has been challenging. The disclosed artificial intelligence can advantageously use facial images to accurately detect a variety of biological traits, including, without limitation, age, gender, weight, height, smoking habits, and alcohol consumption. The disclosed artificial intelligence can also be applied to quantitatively measure important clinical parameters, including, without limitation, uric acid, ALT, hemoglobin, obesity, diabetes, metabolic syndrome, hyperuricemia, NAFLD, and anemia.

The ability to measure human biological traits, such as aging, weight, and height, and identify their modifying factors using 3D facial images has important implications in many fields, including, without limitation, disease prevention and treatment and the healthy extension of life. The disclosed artificial intelligence can accurately predict biological age, which is modified by lifestyle factors such as smoking and alcohol consumption. This provides a foundation for investigating factors that impact aging acceleration, as well as identifying therapeutic interventions that can retard the aging process.

When developing AI algorithms, one of the most important factors to consider is its applicability in a variety of settings. The diagnostic capabilities of the disclosed artificial intelligence are not only applicable to 3D facial images obtained using professional cameras, but are equally applicable to 3D facial images captured using smartphones, thereby demonstrating generalizability. Furthermore, the disclosed artificial intelligence could provide a non-invasive, high-throughput, low-cost, early diagnostic, health screening tool for a variety of common diseases at a point of care or home. The disclosed artificial intelligence can be used to predict any systemic disease that is manifested in human faces, possibly beyond the observational powers of human experts.

### 10. REFERENCES

The following references have been referred to herein, and are all incorporated herein by reference as if set forth in full:
Ackers, I., and Malgor, R. (2018). Interrelationship of canonical and non-canonical Wnt signalling pathways in chronic metabolic diseases. Diabetes & vascular disease research 15, 3-13.
Alberti, K. G., Eckel, R. H., Grundy, S. M., Zimmet, P. Z., Cleeman, J. I., Donato, K. A., Fruchart, J. C., James, W. P., Loria, C. M., Smith, S. C., Jr., et al. (2009). Harmonizing the metabolic syndrome: a joint interim statement of the International Diabetes Federation Task Force on Epidemiology and Prevention; National Heart, Lung, and Blood Institute; American Heart Association; World Heart Federation; International Atherosclerosis Society; and International Association for the Study of Obesity. Circulation 120, 1640-1645.
Alberti, K. G., and Zimmet, P. Z. (1998). Definition, diagnosis and classification of diabetes mellitus and its complications. Part 1: diagnosis and classification of diabetes mellitus provisional report of a WHO consultation. Diabetic medicine: a journal of the British Diabetic Association 15, 539-553.
Cane, J. M., and McCormick, C. M. (2008). In your face: facial metrics predict aggressive behaviour in the laboratory and in varsity and professional hockey players. Proceedings Biological sciences 275, 2651-2656.
Chong, J., Soufan, O., Li, C., Caraus, I., Li, S., Bourque, G., Wishart, D. S., and Xia, J. (2018). MetaboAnalyst 4.0: towards more transparent and integrative metabolomics analysis. Nucleic acids research 46, W486-W494.
Claes, P., Liberton, D. K., Daniels, K., Rosana, K. M., Quillen, E. E., Pearson, L. N., McEvoy, B., Bauchet, M., Zaidi, A. A., Yao, W., et al. (2014). Modeling 3D facial shape from DNA. PLoS genetics 10, e1004224.
Claes, P., Roosenboom, J., White, J. D., Swigut, T., Sero, D., Li, J., Lee, M. K., Zaidi, A., Mattern, B. C., Liebowitz, C., et al. (2018). Genome-wide mapping of global-to-local genetic effects on human facial shape. Nature genetics 50, 414-423.
Clements, A., and Zhang, H. (2006). Minimum Ratio Contours on Surface Meshes, Vol 2006.
Coetzee, V., Perrett, D. I., and Stephen, I. D. (2009). Facial adiposity: a cue to health? Perception 38, 1700-1711.
Cohen, L. (2006). Minimal Paths and Fast Marching Methods for Image Analysis.
Contrepois, K., Jiang, L., and Snyder, M. (2015). Optimized Analytical Procedures for the Untargeted Metabolomic Profiling of Human Urine and Plasma by Combining Hydrophilic Interaction (HILIC) and Reverse-Phase Liquid Chromatography (RPLC)—Mass Spectrometry. Molecular & cellular proteomics: MCP 14.
Cooney, M. T., Dudina, A., De Bacquer, D., Fitzgerald, A., Conroy, R., Sans, S., Menotti, A., De Backer, G., Jousilahti, P., Keil, U., et al. (2009). How much does HDL cholesterol add to risk estimation? A report from the SCORE Investigators. European journal of cardiovascular prevention and rehabilitation: official journal of the European Society of Cardiology, Working Groups on Epidemiology & Prevention and Cardiac Rehabilitation and Exercise Physiology 16, 304-314.
Dehghan, A., van Hoek, M., Sijbrands, E. J., Hofman, A., and Witteman, J. C. (2008). High serum uric acid as a novel risk factor for type 2 diabetes. Diabetes care 31, 361-362.
Dudina, A., Cooney, M. T., Bacquer, D. D., Backer, G. D., Ducimetiere, P., Jousilahti, P., Keil, U., Menotti, A., Njolstad, I., Oganov, R., et al. (2011). Relationships between body mass index, cardiovascular mortality, and risk factors: a report from the SCORE investigators. European journal of cardiovascular prevention and rehabilitation: official journal of the European Society of Cardiology, Working Groups on Epidemiology & Prevention and Cardiac Rehabilitation and Exercise Physiology 18, 731-742.
Efron, B. (1992). Bootstrap Methods: Another Look at the Jackknife. In Breakthroughs in Statistics: Methodology and Distribution, S. Kotz, and N. L. Johnson, eds. (New York, NY: Springer New York), pp. 569-593.
Fagertun, J., Harder, S., Rosengren, A., Moeller, C., Werge, T., Paulsen, R. R., and Hansen, T. F. (2014). 3D facial landmarks: Inter-operator variability of manual annotation. BMC medical imaging 14, 35.
Ferry, Q., Steinberg, J., Webber, C., FitzPatrick, D. R., Ponting, C. P., Zisserman, A., and Nellaker, C. (2014). Diagnostically relevant facial gestalt information from ordinary photos. eLife 3, e02020.
Fishler, M., and Bolles, R. (1980). Random sample consensus: A paradigm for model fitting applications to image analysis and automated cartography. Proc Image Understanding Workshop, 71-88.
Goff, D. C., Jr., Lloyd-Jones, D. M., Bennett, G., Coady, S., D'Agostino, R. B., Gibbons, R., Greenland, P., Lackland, D. T., Levy, D., O'Donnell, C. J., et al. (2014). 2013 ACC/AHA guideline on the assessment of cardiovascular risk: a report of the American College of Cardiology/American Heart Association Task Force on Practice Guidelines. Circulation 129, S49-73.
Grundy, S. M., Cleeman, J. I., Daniels, S. R., Donato, K. A., Eckel, R. H., Franklin, B. A., Gordon, D. J., Krauss, R. M., Savage, P. J., Smith, S. C., Jr., et al. (2005). Diagnosis and management of the metabolic syndrome: an American Heart Association/National Heart, Lung, and Blood Institute Scientific Statement. Circulation 112, 2735-2752.
Gurovich, Y., Hanani, Y., Bar, O., Nadav, G., Fleischer, N., Gelbman, D., Basel-Salmon, L., Krawitz, P., Kamphausen, S., Zenker, M., et al. (2019). Identifying facial phenotypes of genetic disorders using deep learning. Nature Medicine 25.
Haselhuhn, M. P., and Wong, E. M. (2012). Bad to the bone: facial structure predicts unethical behaviour. Proceedings Biological sciences 279, 571-576.
He, K., Zhang, X., Ren, S., and Sun, J. (2016). Deep Residual Learning for Image Recognition.
Henderson, A. J., Holzleitner, I. J., Talamas, S. N., and Perrett, D. I. (2016). Perception of health from facial cues. Philosophical transactions of the Royal Society of London Series B, Biological sciences 371.
Huang, G., Mattar, M., Berg, T., and Learned-Miller, E. (2008). Labeled Faces in the Wild: A Database for Studying Face Recognition in Unconstrained Environments. Tech rep.
Jia, L., Zhang, W., and Chen, X. (2017). Common methods of biological age estimation. Clinical interventions in aging 12, 759-772.
Kaesemodel Pontes, J., Jr, A., Fookes, C., and Koerich, A. (2015). A Flexible Hierarchical Approach For Facial Age Estimation Based on Multiple Features. Pattern Recognition 54.
Kermany, D. S., Goldbaum, M., Cai, W., Valentim, C. C. S., Liang, H., Baxter, S. L., McKeown, A., Yang, G., Wu, X., Yan, F., et al. (2018). Identifying Medical Diagnoses and Treatable Diseases by Image-Based Deep Learning. Cell 172, 1122-1131 e1129.
Kingma, D., and Ba, J. (2014). Adam: A Method for Stochastic Optimization. International Conference on Learning Representations.
Lambin, P., Rios-Velazquez, E., Leijenaar, R., Carvalho, S., van Stiphout, R. G., Granton, P., Zegers, C. M., Gillies, R., Boellard, R., Dekker, A., et al. (2012). Radiomics: extracting more information from medical images using advanced feature analysis. European journal of cancer 48, 441-446.
Lin, T.-Y., Goyal, P., Girshick, R., He, K. & Dollar, P. Focal loss for dense object detection. In Proceedings of the IEEE International Conference on Computer Vision, 2980-2988 (2017).
Masuo, K., Kawaguchi, H., Mikami, H., Ogihara, T., and Tuck, M. L. (2003). Serum uric acid and plasma norepinephrine concentrations predict subsequent weight gain and blood pressure elevation. Hypertension 42, 474-480.
Oh, J., Won, H. Y., and Kang, S. M. (2009). Uric acid and cardiovascular risk. The New England journal of medicine 360, 539-540; author reply 540-531.
Pan, H., Han, H., Shan, S. & Chen, X. Mean-variance loss for deep age estimation from a face. In Proceedings of the IEEE Conference on Computer Vision and Pattern Recognition, 5285-5294 (2018).
Paszke, A., Gross, S., Massa, F., Lerer, A., Bradbury, J., Chanan, G., Killeen, T., Lin, Z., Gimelshein, N., Antiga, L., et al. (2019). PyTorch: An Imperative Style, High-Performance Deep Learning Library.
Poplin, R., Varadarajan, A., Blumer, K., Liu, Y., McConnell, M., Corrado, G., Peng, L., and Webster, D. (2018). Predicting Cardiovascular Risk Factors from Retinal Fundus Photographs using Deep Learning. Nature Biomedical Engineering 2.
Pound, N., Penton-Voak, I., and Brown, W. (2007). Facial symmetry is positively associated with self-reported extraversion. Personality and Individual Differences 43, 1572-1582.
Ricanati, E. H., Golubic, M., Yang, D., Saager, L., Mascha, E. J., and Roizen, M. F. (2011). Mitigating preventable chronic disease: Progress report of the Cleveland Clinic's Lifestyle 180 program. Nutrition & metabolism 8, 83.
Roizen, N. J., and Patterson, D. (2003). Down's syndrome. Lancet 361, 1281-1289.
Rothe, R., Timofte, R., and Van Gool, L. (2015). DEX: Deep EXpectation of Apparent Age from a Single Image.
Saklayen, M. G. (2018). The Global Epidemic of the Metabolic Syndrome. Current hypertension reports 20, 12.
Taigman, Y., Yang, M., Ranzato, M. A., and Wolf, L. (2014). DeepFace: Closing the Gap to Human-Level Performance in Face Verification.
Tamura, H., Mori, S., and Yamawaki, T. (1978). Textural Features Corresponding to Visual Perception. Systems, Man and Cybernetics, IEEE Transactions on 8, 460-473.
Valentine, M., Bihm, D. C. J., Wolf, L., Hoyme, H. E., May, P. A., Buckley, D., Kalberg, W., and Abdul-Rahman, O. A. (2017). Computer-Aided Recognition of Facial Attributes for Fetal Alcohol Spectrum Disorders. Pediatrics 140.
Verdin, E. (2015). NAD(+) in aging, metabolism, and neurodegeneration. Science 350, 1208-1213.
Welker, K., Goetz, S., and Cane, J. (2015). Perceived and experimentally manipulated status moderate the relationship between facial structure and risk-taking. Evolution and Human Behavior 36.
Zhang, K., Liu, X., Shen, J., Li, Z., Sang, Y., Wu, X., Zha, Y., Liang, W., Wang, C., Wang, K., et al. (2020). Clinically Applicable AI System for Accurate Diagnosis, Quantitative Measurements, and Prognosis of COVID-19 Pneumonia Using Computed Tomography. Cell.
Zhao, Q., Shi, C. Z., and Luo, L. P. (2014). Role of the texture features of images in the diagnosis of solitary pulmonary nodules in different sizes. Chinese journal of cancer research=Chung-kuo yen cheng yen chiu 26, 451-458.
Zhu, X. H., Lu, M., Lee, B. Y., Ugurbil, K., and Chen, W. (2015). In vivo NAD assay reveals the intracellular NAD contents and redox state in healthy human brain and their age dependences. Proceedings of the National Academy of Sciences of the United States of America 112, 2876-2881.

### Certain Non-Limiting Embodiments

### Summary

The human face is a multipartite trait composed of distinct features that vary significantly among individuals. Using a 3D camera and deep learning, we developed an AI model on representation of 3D facial quantitative features and applied it to assess biometric features, lifestyles factors, and five metabolic diseases, which all achieved good performances. We then showed its feasibility in a “point-of-care” setting. We identified a number of metabolites in 12 known metabolic pathways associated with both facial features and Type 2 diabetes mellitus, providing a framework linking the disease to the face features through the underlying biochemical mechanisms. The identification of facial phenotypic features associated with both biometric and metabolic parameters and its potential applications in both biological research and clinical applications opens the door for a new scientific dimension based on 3D face phenotypical features and should have a broad impact in biology and medicine.

### Introduction

With advancements in global economics and healthcare systems, the top causes of death have shifted from infectious diseases to chronic diseases (Ackers and Malgor, 2018; Ricanati et al., 2011), with metabolic diseases, including obesity, type 2 diabetes mellitus (T2DM), and non-alcoholic fatty liver disease (NAFLD), rapidly increasing in prevalence and overall mortality (Alberti et al., 2009). Diabetes is an extremely common disease currently affecting 382 million individuals worldwide, and is predicted to afflict 629 million by 2045 (Cho et al., 2018). In addition, NAFLD affects as many as one billion people worldwide (Loomba and Sanyal, 2013). The actual prevalence is considerably higher, with a large number of patients remaining undiagnosed, untreated, and unaware of their illness and its long-term health consequences (Herman et al., 2015). Lifestyle factors such as smoking and excessive alcohol use are two of the major known contributing factors but also modifiable risk factors for metabolic diseases. Early intervention is crucial in reducing the disease burden imposed by metabolic diseases, yet detection is challenging. Therefore, there is a pressing need to develop new technologies which can diagnose metabolic diseases in a non-invasive and cost-effective manner.

The human face is a multipartite trait composed of distinct physical features (eyes, nose, chin, mouth and forehead), in which their size, shape and composition are distinct and show variations among individuals (Claes et al., 2018). Physicians have been using facial appearance and expression to assess a patient's health status since ancient times. Facial features associated with inherited syndromes have highly recognizable facial characteristics that are very informative for physicians (Roizen and Patterson, 2003). The facial features of neurologic diseases such as a mask-like expression in Parkinson's disease are well described. Unique and uneven distribution of physical signs including jaundice, xanthelasma, spider nevi, telangiectasia, and certain patterns of pigmentation including café au lait spots, are also well documented. For example, xanthelasma tends to accumulate on or around the eyelids on the medial side, suggesting that the location for the deposit of cholesterol is not a random event. Looking back into the history of medicine, both Western and Chinese Medicine had placed a lot of emphases on the recognition of clinical features for the diagnose of diseases, followed by confirmation from further investigations. However, for subtle changes or for those combinatorial and non-linear changes, their detection scope was limited by the observational ability and the comprehension of most of the clinicians.

Modern technology has allowed for the measurement and recording of parameters beyond the scope of human physicians (e.g. fine measurements of shape and surface texture), enabling the construction of models that can evaluate multiple parameters and subtle differences that can allow the identification of new clinical signs/parameters of diagnostic and prognostic significance. Advances in artificial intelligence (AI) have inspired innovations and applications in many healthcare areas (He et al., 2019; Kermany et al., 2018; Topol, 2019; Zhang et al., 2020). In combination with high resolution digital cameras, facial analysis using AI has performed extremely well in the realms of facial recognition and personal verification (Huang et al., 2008), and deep learning has been applied successfully to the characterization of human facial parameters and their associations with personality traits (Welker et al., 2015). Based on the above discussion, we hypothesized that by using a high-resolution 3D camera to capture the fine facial features, it is possible to train an accurate AI system based on facial parameters that correlate well with the biological and metabolic status of individuals.

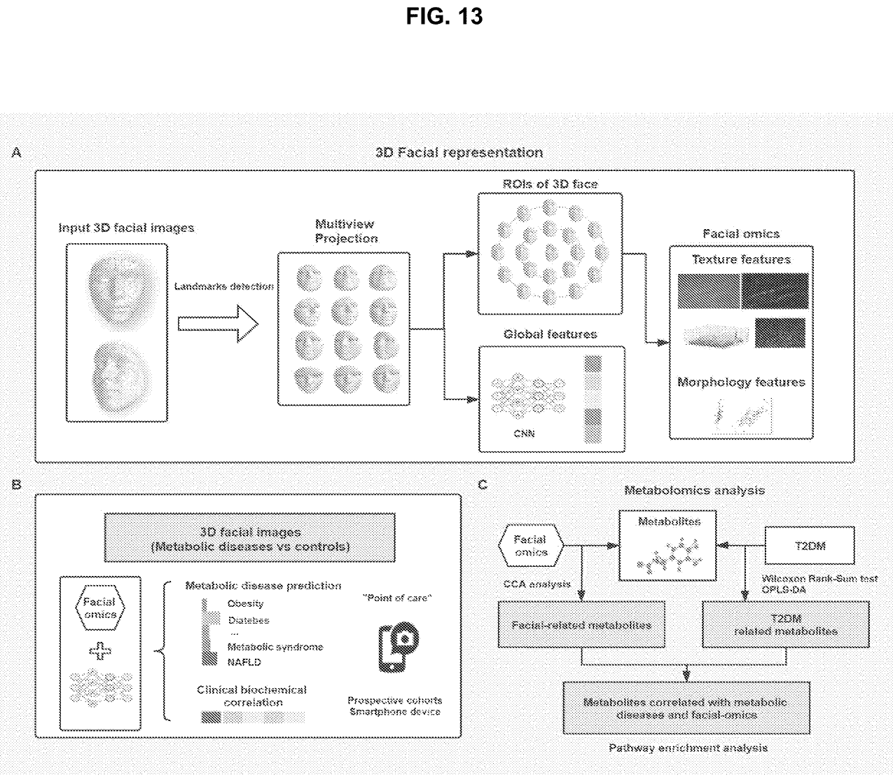

This study was designed as a proof of principle study. First, as a proof-of-concept, metabolic parameters were chosen as our first attempt since these endpoints are dynamic and measurable and have a high probability to be revealed in the facial phenotypic markers. We established an AI system that projects basic biometric parameters, including age, gender, height, body weight, body mass index (BMI), based on 3D facial features. Then, we investigate the impact of life-style habits including smoking and excessive alcohol use on AI's prediction. Moreover, an attempt was made to establish an AI system for prediction on common metabolic disease using 3D facial features (FIGS. 13A and 13B).

As many subtle facial parameters with metabolic diseases/pathways may have not been explored due to the limits of our observational ability, experience as well as measurement tools, we further investigated whether metabolic diseases and 3D face may share similar structural and pathophysiological pathways using a metabolomics approach. Metabolomics has been employed to identify metabolites that are associated with a particular physiological conditions such as acute exercise or processes such as pregnancy(Contrepois et al., 2020; Liang et al., 2020). Metabolomics, which is broadly acknowledged to be the omics discipline that is closest to the phenotype, can also be used to identify metabolites that could alter a cell or an organism's phenotype (Guijas et al., 2018; Johnson et al., 2016). We set out to identify metabolites linking metabolic diseases and facial-omics (morphology feature and texture feature) and the underlying biochemical pathways (FIG. 13C).

If we can connect facial phenotypic features with the biometric or metabolic parameters, this may provide the foundation for a new discipline of science that uses electronic and AI technology to mine the “undiscovered” features in the advancement of clinical sciences from a different approach. Finally, with the latest 3D camera development, we also evaluated whether 3D facial parameter assessment can be conducted in a point-of-care setting using a smartphone.

### Results

### Patient Characteristics and Study System Overview

The general scheme of our study design and procedures are described in FIG. 13. A well-established dataset from the China Consortium of 3D Facial Image Investigation (CC-3DF) dataset was utilized in this study. These were subjects followed up longitudinal for regular health check starting with a cross-sectional study back in 2013 (Wang et al., 2016). If they consented to this study, they were selected whether they participate with the 3D face scanning and the use of the medical record data. After their fasting blood draws and medical follow-up, 7,221 subjects consented to have their 3D facial images taken using a 3dMDface camera system (www.3dmd.com) for the AI model development. For each subject from the CC-3DF cohort consented to this study, medical records of clinical information were collected including demographic information, life-style (including smoking, alcohol use), routine physical examination, and clinical laboratory (FIG. 13, Table 1 and Supplemental Table 1). After the establishment and validation of this model, the 3D images technology had evolved to an extent that it was included in a smartphone setting. To determine the general applicability of our AI model, we also conducted another prospective “point of care” study using 3D facial images from another cohort of 432 patients in Guangzhou using a pre-defined smartphone in the clinical evaluation setting. Demographics and clinical parameters of all the study subjects are provided in Table 1 and Supplemental Table 1.

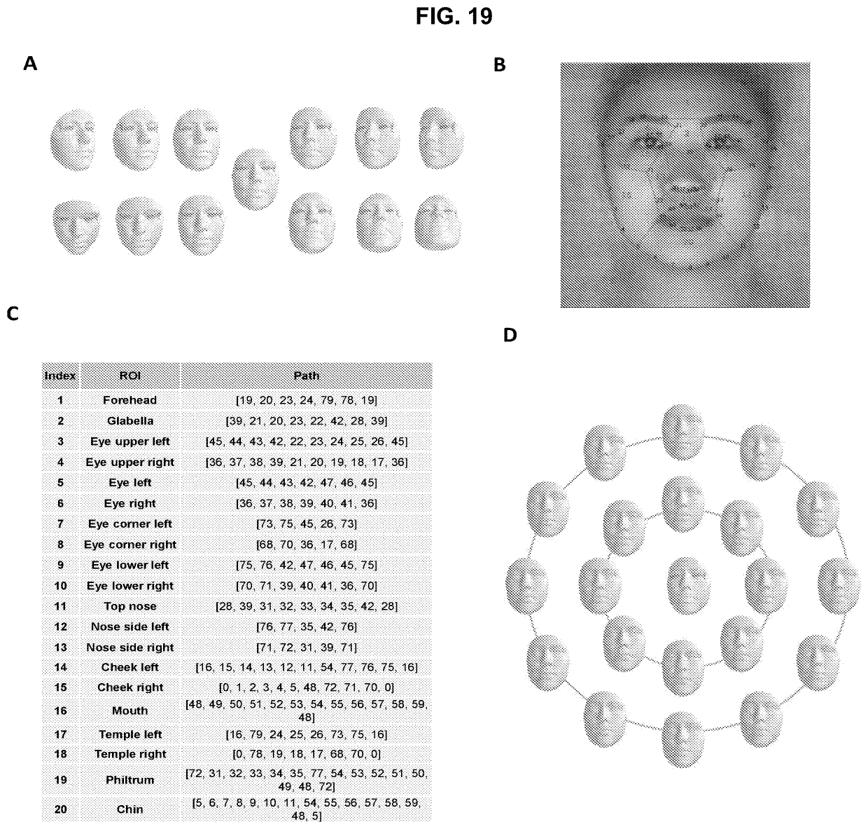

A schematic illustration of the proposed AI model is presented in FIG. 13. With the input of a 3D facial image, an automated preprocessing pipeline was developed, which included landmark detection, standardization, rotation, and projection to multi-views of directions (FIG. 19A and details were provided in Methods). To characterize each facial image, the AI model extracted the global features and local features for the 3D face representation. The global feature extraction employed a deep convolutional neural network (DCNN) to obtain the global information on a 3D multi-view facial image. The local feature extraction (or facial-omics extraction) entailed a high-throughput extraction of quantitative features of a facial image, which constituted “the 3D facial-omics” (FIG. 19B-D and details provided in Methods). Based on the region of interests (ROIs) of 3D facial segments defined by landmarks, the proposed “facial-omics” included quantification of both the morphology and the texture features. The morphology features measured the shape and spatial relationships. The texture features represented the local brightness, structure patterns, or the spatially repetitive structure of surfaces such as local variations of scale, orientations, or other geometric characteristics, which were important visual patterns of facial components (Kaesemodel Pontes et al., 2015). For further enhancement, a joint model that integrated both the global features and facial-omics was developed (See Methods). AI Model training and evaluation were based on k-fold cross-validation (k=10). Accordingly, the dataset was split into mutually exclusive sets for training/validation (90%) and for testing (10%). This process was repeated 10 times, yielding a collectively exhaustive test set.

For the 551 subjects who consented for additional fasting blood draws in order to participate on metabolic profiling, their plasma samples were subjected to liquid chromatography followed by mass spectroscopy studies for metabolite features. In the initial screening, 3,560 metabolite features were identified. The 3,560 metabolite features were subject to simultaneous analyses of associations with facial-omics and T2DM. Importantly, 20 metabolites that highly correlated with facial-omics and differentially present in T2DM were annotated and used for a pathway enrichment analysis (FIG. 13C).

Correlation with Biometric Parameters (Age, Gender, Height, Body Weight and BMI)

First, we tested the ability of our AI models to associate basic biometric parameters, including age, gender, height, body weight and BMI, from 3D facial images. As advanced age is a known risk factor for metabolic diseases and effects of aging is known to be reflected in facial features, we first attempted to establish an AI model in associating the 3D facial features with chronological age. Once all aging features captured by 3D facial scanning reflected through AI were established, the age established by AI (i.e., FaceAge) ideally should reflect the biological status of an individual. This means that if there is a significant difference between a person's FaceAge and chronological age, e.g. the model predicts a person to be older than he/she actually is, there could be relevant clinical implications.

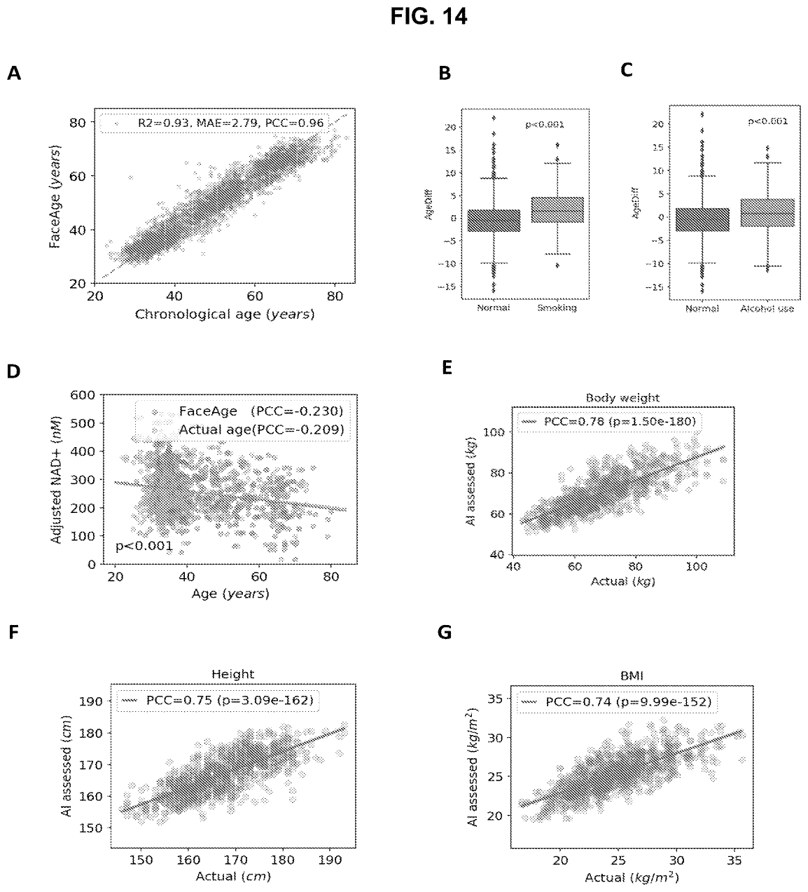

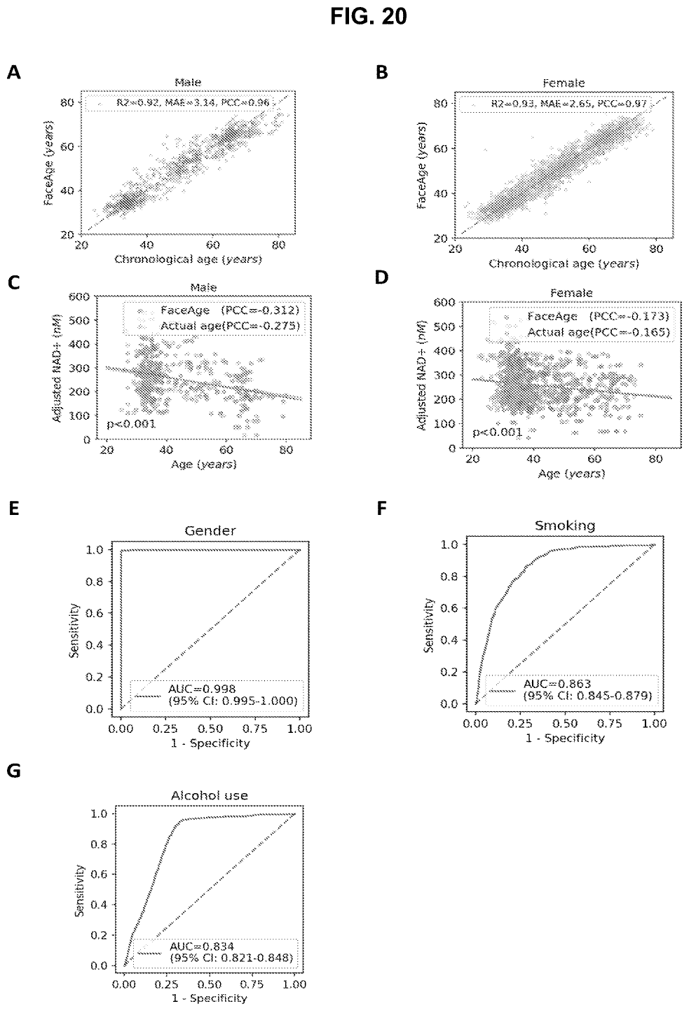

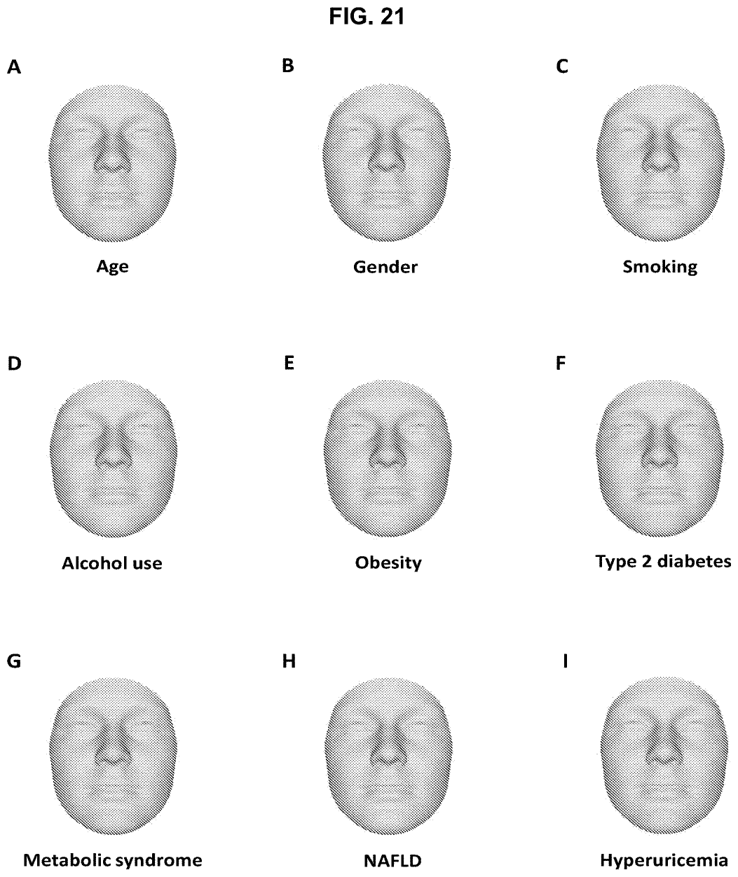

7,221 3D facial images were used for AI model development for age assessment (90% for training/validation, 10% hold out for testing). The association between predicted FaceAge and true chronological age was further evaluated using a Pearson's correlation analysis. (FIG. 14A, FIGS. 20A and 20B). The predicted age and chronological age had a strong linear relationship, with a Pearson correlation coefficient (PCC) of 96%, coefficient of determination (R2) of 0.93, and a mean absolute error (MAE) of 2.79 years (FIG. 14A). These results show that our AI model can assess chronological age with high precision. To make the AI system decision more interpretable, we generated a “heatmap” to highlight the skin regions of the 3D face relevant to the predictions of the model (see more details in Methods). Review of the AI algorithm showed that local features in the peri-orbital areas and cheek were the drivers for the overall AI assessment (FIG. 21A).

We also evaluated the AI model ability to determine the gender of the subject. Our AI model was able to distinguish between males and females with an Area under the Curve of ROC (AUC-ROC) of 0.998 (95% CI: 0.995 to 1.000) (FIG. 20E). Review of the AI algorithm showed that the features on areas included peri-oral and cheek areas were the main drivers of the overall AI assessment (FIG. 21B). Review of the subjects with wrong gender assignment showed that they were having very neutral features even to the eyes of the clinicians.

Next, we assessed whether 3D facial features correlated with body weight. FIG. 14E showed that through deep learning, an AI algorithm based on 3D facial features could be established which showed an excellent correlation with body weight (PCC=0.78, p=1.50E-180). FIG. 14F showed that a very strong correlation could also be established for body height (PCC=0.75, p=3.09E-162). With the strong correlations in body weight and body height, it was not surprising that our AI model based on 3D facial features could provide an accurate assessment of body mass index (BMI), which is a composite of the body weight in kilograms relative to the square of the body height in meters (FIG. 14G, PCC=0.74, p=9.99E-152).

Correlation with Life-Style Factors (Smoking and Excessive Alcohol Consumption)

It is well known that life-style factors can impact the aging process. Next, we investigated whether smoking and alcohol consumption could modify the predicted chronological age (or biological age). As the first step, we trained our AI model on 5,463 subjects with no habits of smoking and alcohol consumption according to their medical records. Then, we applied the trained model to determine FaceAge for the 967 subjects with a habit of smoking (on average of more than one pack (20 cigarettes)/day for at least one year) and 1,406 subjects with a habit of excessive alcohol consumption (defined as having an average of 60 ml or more of alcohol per day for man and an average of 30 ml or more per day for women).

The AgeDiff is defined by the difference of the AI determined FaceAge and the actual chronological age. Smoking significantly increases FaceAge across the age spectrum, with an average AgeDiff of 1.79 years (P<0.001, FIG. 14B). Similarly, excessive alcohol consumption significantly increases the AI determined age, with an average AgeDiff of 0.88 years (P<0.001, FIG. 14C). For the next step, we investigated whether we could determine the lifestyle factors, smoking and alcohol use (as defined above, which has definitive endpoints) directly through our AI model. The AI model achieved a good prediction accuracy for the smoking status, with an AUC-ROC of 0.863 (95% CI: 0.845 to 0.879) (FIG. 21F). When an attempt was made to associate smoking with region of interests (ROIs) in the facial map, the information on the cheek region captured by the AI model was one of the key drivers for the assessment (FIG. 21C). Similarly, our AI model achieved a good prediction accuracy for chronic excessive alcohol use status, with an AUC-ROC of 0.834 (95% CI: 0.821 to 0.848) (FIG. 20G). Chronic excessive alcohol localized to the cheek and chin regions in the facial ROI map (FIG. 21D).

Analysis of Plasma NAD+ with Biological and Chronologic Age

To study the underlying molecular mechanism of aging, we studied metabolic pathways that have previously been implicated in the aging process. Metabolic pathways have been implicated in the aging process. Depletion of nicotinamide adenine dinucleotide (NAD+), an essential electron transporter in mitochondrial respiration and oxidative phosphorylation, was previously shown to correlate with aging in both rodents and humans, (Verdin, 2015; Zhu et al., 2015). NAD+ is the sole substrate for the nuclear repair enzyme, poly (ADP-ribose) polymerase (PARP) and the sirtuin family of NAD-dependent histone deacetylases, both of which are essential in the regulation of the aging process.

We were able to analyze NAD+ levels in blood samples from 806 healthy controls (mean age of 41.44±12.22 years). Both gender and body mass index (BMI), known confounding factors for NAD+ levels (Clement et al., 2019), were adjusted in the multivariate regression model with age (actual age vs FaceAge) for correlation analysis of NAD+(see more details in Methods). There was a significant negative correlation between in NAD+ levels and aging (in both chronological age and facial age, FIG. 14D, FIGS. 14C and 14D). Our AI model based on FaceAge achieved a PCC with NAD+ of −0.312 in male and −0.173 in female, which was better than the correlation with the actual chronological age with a PCC of −0.275 in male and −0.165 in female. Our AI model correlation with NAD+ is superior to the correlation between chronological age for both male and female (p<0.001).

### Prediction for Metabolic Diseases

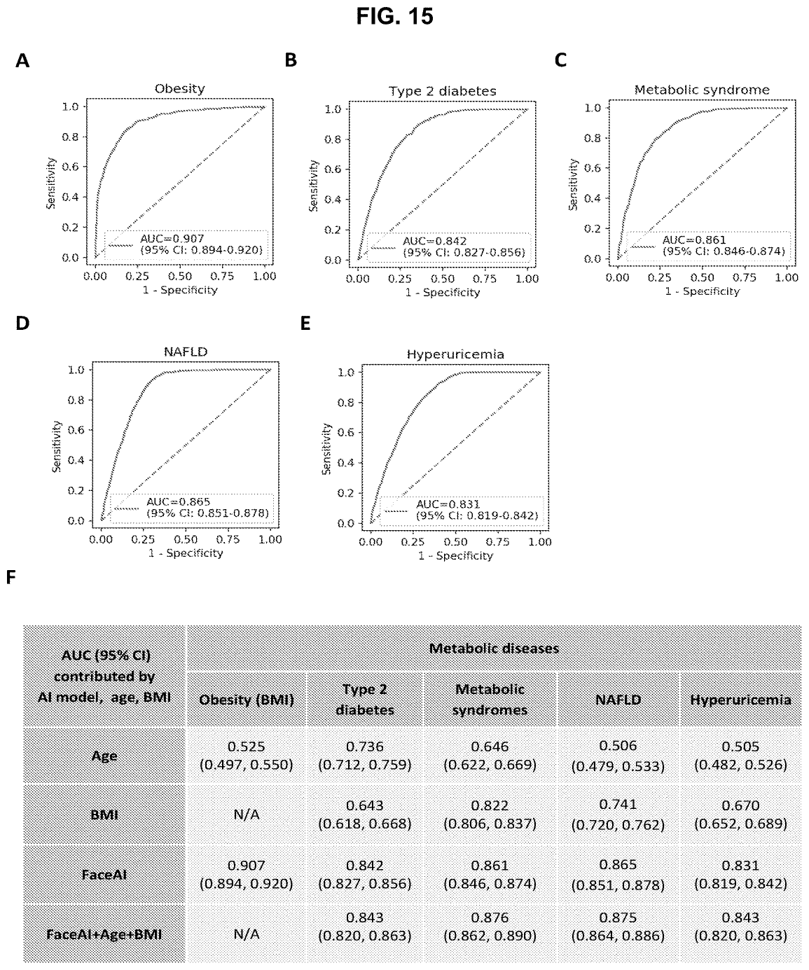

Next, we trained AI model to take facial-omics features and facial global features as an input, and made multiple predictions on metabolic diseases, including obesity, Type 2 diabetes mellitus (T2DM), metabolic syndrome, NAFLD and hyperuricemia. We also analyzed age and BMI as risk factors to determine their contribution in prediction of metabolic diseases compared to the 3D facial assessments. The AI model showed a good prediction accuracy for obesity (as defined by BMI >30), with an AUC-ROC of 0.907 (95% CI: 0.894-0.920, FIG. 15A). Similarly, the AI model achieved a good prediction accuracy for T2DM, with an AUC-ROC of 0.842 (95% CI:0.827-0.856) (FIG. 15B). Our AI model also showed a good prediction performance for the metabolic syndrome with an AUC-ROC of 0.861 (95% CI: 0.846-0.874) (FIG. 15C). We then tested the AI system's ability to predict another metabolic-related disease nonalcoholic fatty liver disease (NAFLD). NAFLD is associated with metabolic risk factors such as T2DM, and patients with known excessive alcohol use were first excluded from the analysis. With this approach, our AI model showed a good prediction of NAFLD with an AUC-ROC of 0.865 using facial features (95% CI: 0.851-0.878) (FIG. 15D). Lastly, as hyperuricemia is a part of metabolic de-arrangement and often associated with obesity (Masuo et al., 2003) and T2DM (Dehghan et al., 2008), we trained the AI model to identify hyperuricemia. With our AI model, the prediction of hyperuricemia obtained an AUC-ROC of 0.831 (95% CI: 0.819-0.842) with facial features (FIG. 15E).

Age and high BMI are well established clinical risk factors for metabolism-related disease (Jura and Kozak, 2016). Therefore, to ensure that our AI model were not solely predicting via age and BMI, we first developed baseline prediction models using these risk factors (risk factor-only model). As summarized in FIG. 15F, in all five metabolic diseases assessed, 3D facial features identified by our AI model in our algorithm showed a better predictive performance than that by age and BMI. We next explored the impact of a combination of all three elements (age, BMI and the AI model) on disease prediction. It gave a slight better numerical performance than the AI algorithm alone for metabolic syndrome and hyperuricemia, and gave a comparable performance to AI model alone in diabetes and NAFLD (FIG. 15F). These results suggested that the AI deep learning approach can identify facial features and construct algorithms that 3D face could represent subtle cues derived from metabolic diseases, thus may have good potential in being a part of clinical diagnostic and follow-up management tools in the future.

Next, we attempted to map the different ROIs on 3D face that the AI model utilized in its association with various metabolic diseases (FIGS. 21E-21I). The ROIs for obesity, T2DM, metabolic syndrome, NAFLD, and hyperuricemia were depicted in FIG. 21E to 21I, respectively. It is interesting to note that a number of the conditions showed ROIs in the same area (e.g. cheek and chin areas) but it was the details of the facial-omics features in the ROIs, in conjunction with overall global features that allowed the AI model to capture and manifest for the observed correlation/accuracy.

Association of Facial-Omics with Plasma Metabolites and T2DM

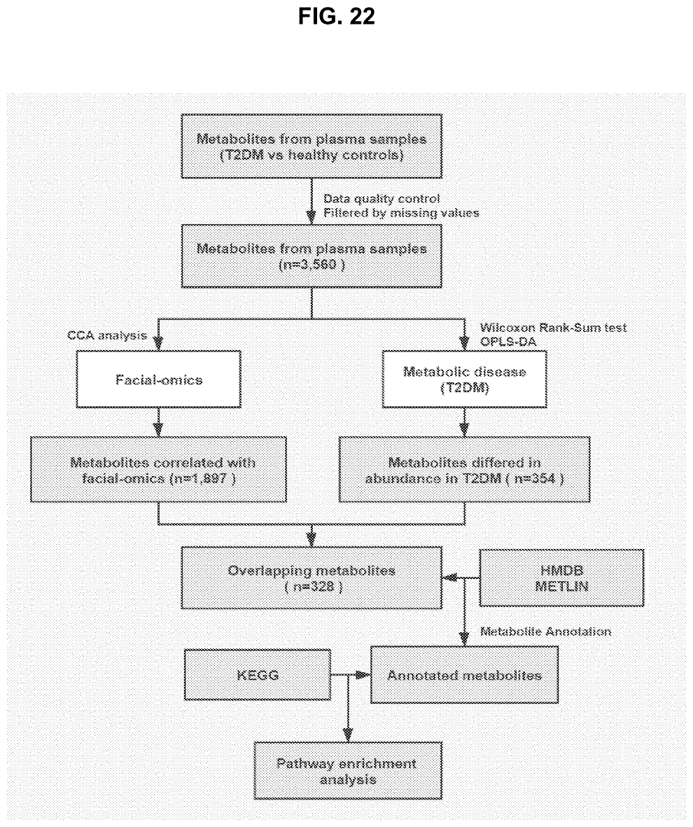

Next, we attempted to identify metabolites that might be associated with 3D facial features and T2DM (FIG. 22). A total of 551 subjects with fasting blood draws and 3D face scanning from subjects with T2DM (n=91) and healthy controls (n=460) were included for metabolomics profiling (FIG. 13C). To remove the impact of obesity as a confounding variable, analysis was performed on non-obese individuals (see more details in Methods). Finally, 3,560 metabolic peaks (features) were detected and selected for further analysis after passing the initial quality control and removing peaks with missing values. In the following study, we performed a simultaneous analysis to map the metabolite features to facial-omics and identify differential metabolites between T2DM versus control groups. (FIG. 22, See more details in Methods).

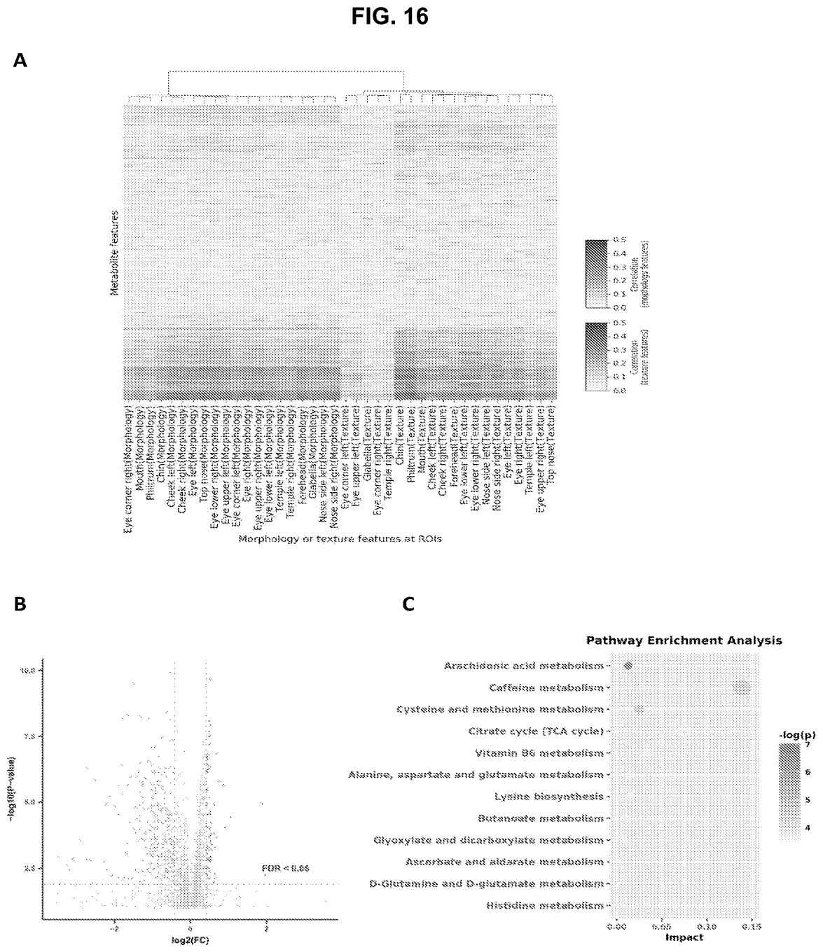

We first map a total of 3,560 metabolite features to facial omics (morphology and texture features at ROI level) through a multivariate canonical correlation analysis (CCA) (FDR<0.01). 1,897 metabolite peaks were identified as facial-associated metabolite biomarkers shown in the heatmap (FIG. 16A).

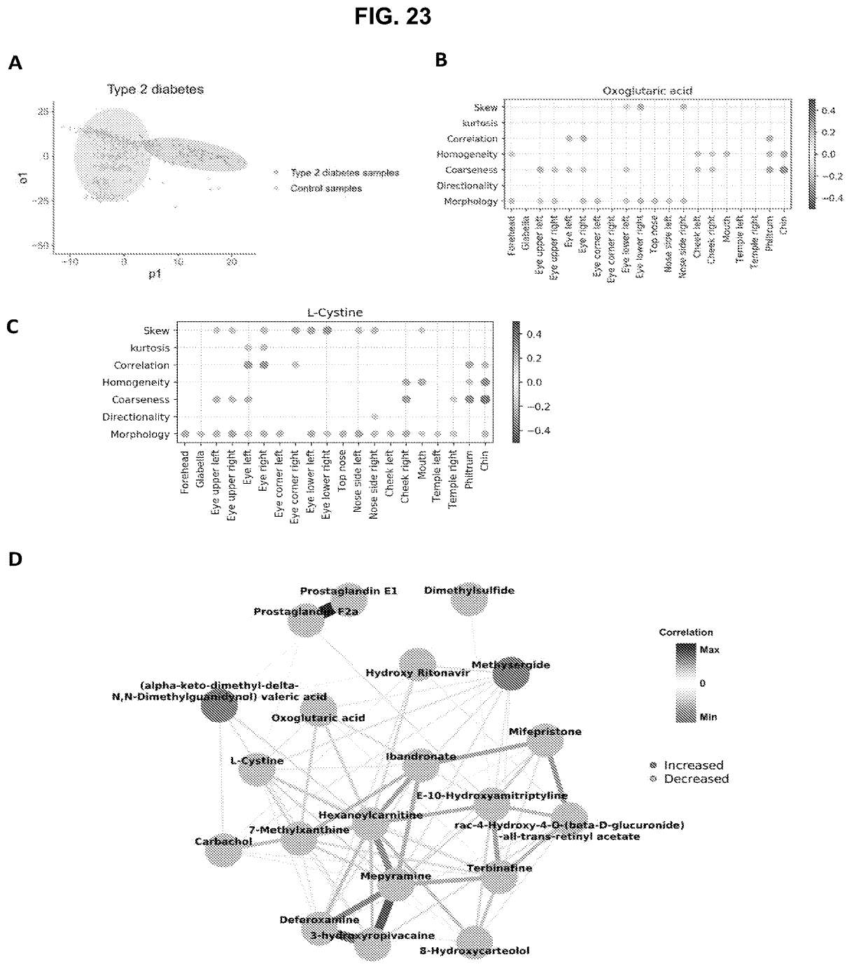

In parallel, we also investigated the differential metabolite features between T2DM and control groups from the initial 3,560 metabolites. To that end, the orthogonal partial least squares discriminant analysis (OPLS-DA) was applied to determine metabolites that could discriminate T2DM from healthy controls (FIG. 23A). We also applied a Wilcoxon test and Fold Change analysis to identify most significant T2DM-related metabolite features from the initial 3,560 candidates (adjusted P-value (FDR)<0.05, Fold Change>1.5 in any direction) (FIG. 16B). A total of 354 metabolite features that markedly changed in abundance between T2DM and controls were identified by the combination of above-mentioned univariate (a volcano plot) and OPLS-DA statistical methods (see more details in Methods).

Next, by comparing T2DM-associated metabolites with that identified through facial-omics, an overlap of 328 metabolite features were selected for further analysis (FIG. 22). To further investigate the underlying pathways of the overlapping metabolite features, we searched the Human Metabolome Database (HMDB) and the METLIN (http://metlin.scripps.edu) to match them to known biochemical metabolites, which led to the identification of 20 known metabolites. The 20 metabolites were then put into the Kyoto Encyclopedia of Genes and Genomes (KEGG) database for a pathway enrichment analysis using the MetaboAnalystR software package (Chong and Xia, 2018). A total of 12 metabolic pathways were identified, including arachidonic acid metabolism, caffeine metabolism, cysteine and methionine metabolism, and tricarboxylic acid (TCA) cycle, etc., which were related to diabetes mellitus (FIG. 16C).

For example, the TCA cycle, one of most essential energy metabolic pathways (Martinez-Reyes and Chandel, 2020), was identified as having a significant impact (FDR <0.05, Global test). The dysregulation of TCA cycle related enzymes and metabolites in mitochondria of pancreatic β-cells has been associated with the pathogenesis of type 2 diabetes (Fex et al., 2018). Specifically, the involved Oxoglutaric acid (also known as α-ketoglutarate), an important metabolic intermediates of TCA cycle, was significantly decreased in type 2 diabetes patients in our analysis (FC=0.55, FDR=1.31E-3), which was consistent with a previous report that it play a protective role in the development of T2DM (Ren et al., 2019).

L-cysteine which was related to cysteine and methionine metabolism, was identified as having a significant impact (FDR=3.20E-04, Global test). The identified L-Cystine is a metabolic source of L-Cysteine. It is reported that a high plasma L-Cysteine level is involved in the deterioration of insulin release of pancreatic beta-cell associated with an increased risk of diabetic conditions (Kaneko et al., 2006). In addition, we found L-Cystine, was significantly decreased (FC=0.421, FDR=3.20E-04), suggesting that the metabolism of L-Cystine was over activated, which might suggest a potential new mechanism of type 2 diabetes.

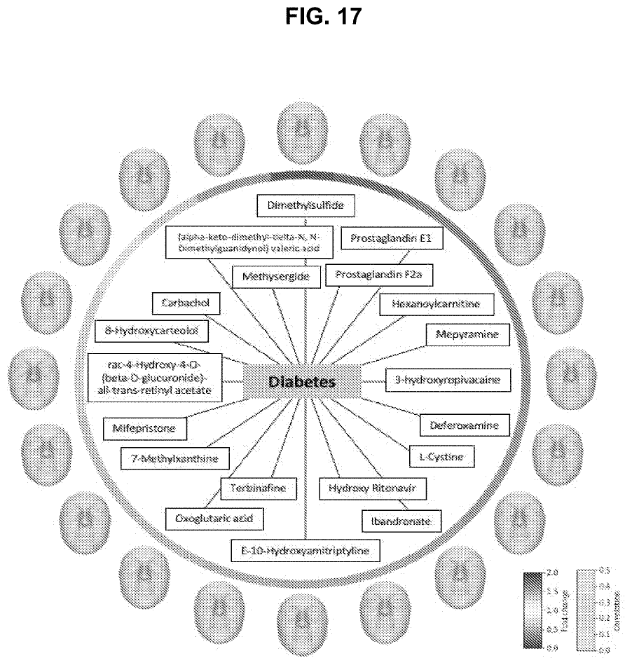

We next performed a correlation analysis of the 20 annotated metabolites mentioned above, which significantly increased or decreased in T2DM patients (FIG. 23C). The findings revealed a highly coordinated metabolic network underlying the altered metabolites linking T2DM and 3D facial features. As we hypothesized that information on metabolism is encoded in the 3D face, we attempted to map these 20 metabolites onto the specific facial-omics parameters. A canonical correlation analysis (CCA) was performed to associate 20 metabolites and morphology/texture features of facial ROIs segments. We visualized the resultant facial projection display. Based on this approach, we were able to map these 20 metabolites to specific ROIs (FIG. 17, results to be discussed in Discussion). As for Oxoglutaric acid and L-Cystine, we further identified their associations with individual facial-omics features (morphology features and texture features) by applying a Pearson's correlation test (FIGS. 23B-23C). Our analysis highlighted the fact that metabolites related to facial features and certain diseases can be identified and their underlying pathways explored.

### General Applicability of Our AI Model in a “Point of Care” Setting Using a Smartphone

Given the latest rapid development of electronic camera and the potential broad appeal of our AI-based model based on non-invasive 3D facial imaging, a 3D image capture and analysis system based on a smartphone camera with a structured light module together with a prototype mobile application software was developed to connect to our AI model server. The server for AI model calculation was established with assurance to Health Insurance Portability and Accountability Act (HIPAA) compliance.

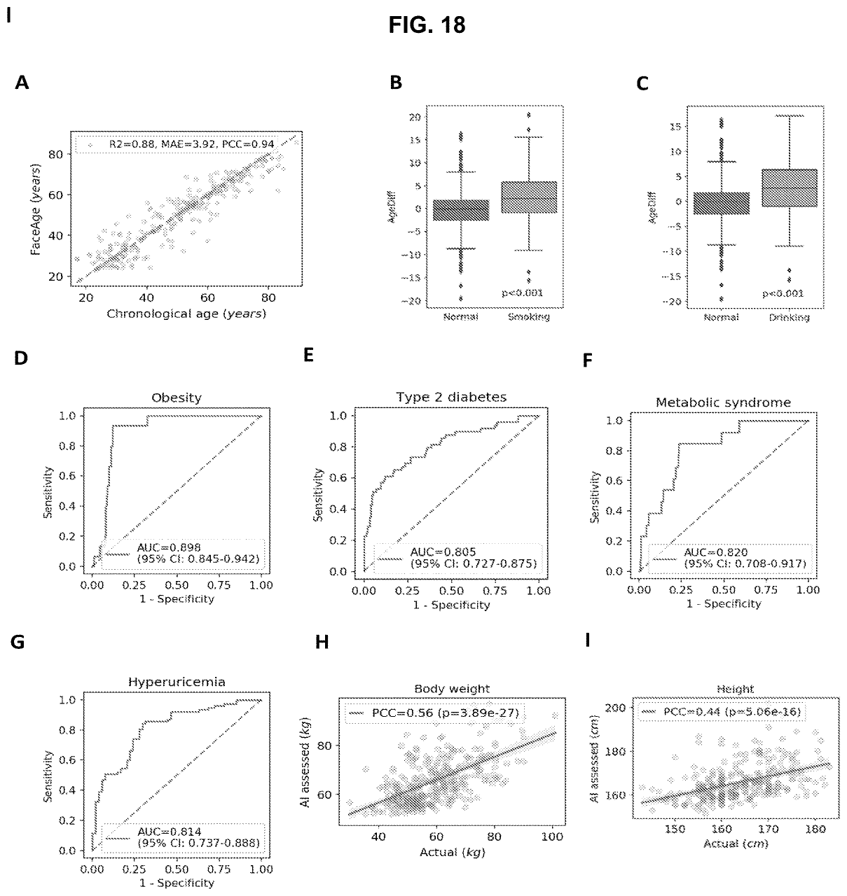

A prospective study was conducted on 432 subjects who attended their annual health check visit in Guangzhou (Supplemental Table 1). With this smartphone-based setting, we attempted to demonstrate the general applicability of this approach. FIG. 18A showed a good correlation between chronological and FaceAge (R2=0.88, PCC=0.94, MAE=3.92). Subjects with lifestyles of smoking or alcohol use also showed an accelerated FaceAge estimation compared to the chronological age (P<0.001) (FIGS. 18B and 18C). In addition, there was a good correlation for the AI assessed body weight (PCC=0.56, p=3.89E-27) and height (PCC=0.44, p=5.06E-16) (FIGS. 18H and 18I).

The AI model also achieved a good predictive performance with AUC of 0.898 (95% CI: 0.845-0.942) for obesity, 0.805 (95% CI: 0.727-0.875) for T2DM, 0.820 (95% CI: 0.708-0.917) for metabolic syndrome, and 0.814 (95% CI: 0.737-0.888) for hyperuricemia (FIGS. 18D-18G). Analysis of NAFLD was not performed in this dataset due to the insufficient number of NAFLD subjects.

### Discussion

This study showed a few important points. First, there are numerous clinically related information embedded subtly in the face, which were revealed with the advancement of electronic image capturing and AI deep learning. Second, this information may have a significant value in biology research as demonstrated by the correlation with various plasma metabolites. Third, this approach may also have an important role in clinical care of subjects with metabolic diseases. Finally, the general applicability of this approach was demonstrated through the adaptation of this approach with the use of smartphone and AI through an internet connection.

AI-assisted deep learning has been successfully applied to facial recognition for person identification. Correlations between craniofacial characteristics and genetic disorders have been discovered in both clinical contexts (Ferry et al., 2014; Valentine et al., 2017) and non-clinical populations (Claes et al., 2014). Clinical signs on the face were well established for many diseases, including metabolic, endocrine, gastrointestinal, liver, neurological, hematological, respiratory and many other systems. Several clinical observations were especially pertinent and worth mentioning. For example, endocrine diseases associated with clinically significant facial features include Cushing syndrome, Addison's disease, and nephrotic syndrome are well described. The differences in Cushing syndrome and obesity in terms facial features is obvious to many physicians. These observations suggest that hormonal and metabolic changes could affect facial features resulting in different patterns. These types of clinically-relevant physical signs were established through decade-long careful observations coupled together with the demonstration of their strong associations with the human diseases. Often the association was recognizable in situations when it was strong enough and only a few parameters are considered in a non-linear correlation. This limitation makes new discoveries very difficult and beyond the scope of the clinical interpretation by most physicians. Therefore, how to develop automated and reproducible analysis methodologies to extract more information from non-invasive facial images is challenging. Here, facial-omics, the high-throughput extraction of large amounts of quantitative and interpretable features (morphology and texture features) from 3D face, promised to address this problem and is one of the approaches that hold great promises but need further validation in multi-centric settings.

With the assistant of AI, it is expected we could assess the biometric and metabolic parameter information from the facial imaging far beyond human resolution. All the complicated parameters/features in human biology were all embedded in the nucleus of a single cell manifested differently in different tissue/physiological context through the interplay between genetic and external factors. Given the high number of clinical physical signs already identified in the face and the complexity of the facial biologic structure, we hypothesized that there is significant undiscovered subtle information embedded in the face that can be extracted with the latest advancements in technology including 3D high resolution camera and the use of AI deep learning. If successful, this is similar to the use of the right tools in the unlocking of knowledge in molecular biology, immunology and structural biology.

As a first step, we hypothesized that the face contains information that correlated strongly with the biometric features, including age, gender, body weight, height, and BMI. These are established endpoints and can serve as a good case to validate our approach. Our AI approach achieved excellent prediction for biological age (FaceAge) which is comparable, if not better than many of other commonly used age estimation/prediction methods, including DNA methylation-based approach (Hannum et al., 2013; Horvath, 2015). In addition, the features identified by our AI system are very similar to how we judge others' age from a clinical angle.

The strong performance on our AI model assessed gender is also not surprising and in fact, the key parameters identified by AI allowed us to appreciate how ourselves, as clinicians, judge on the subjects' gender without realizing the actual process in our brain involved in our pattern recognition. For the subjects with uncertain or wrong gender assignment by our AI model, we have reviewed and found that the facial features of these subjects were very much of neutral gender features based on the eyes of the clinicians. As gender is determined by our genetic makeup and the balance between androgen and estrogen levels in the body, this correlation also highlighted the potential relationship between the balance of sex hormones and facial features/parameters.

Then we explored the value of our AI model on assessing smoking and excessive alcohol use, which have well defined endpoints based on the clinical definitions. The accurate assessment of our AI model based on 3D Face images on these two important lifestyle parameters are worth-noting. We also demonstrated that both smoking and excessive alcohol consumption are associated with a slightly older FaceAge (i.e. biologic age), something that clinicians have long been suspecting. The number of subjects who were both smokers and excessive alcohol drinkers were small, and we were not able to draw a definitive conclusion as to whether the combination of these two lifestyles will lead to further implications to their FaceAge.

With the strong correlation of these biometric features, we explored the correlation with plasma NAD+, a factor that was commonly evaluated with the aging process. There was a stronger correlation of plasma NAD+ with our AI model determined FaceAge than the chronological age. This data indicated that the FaceAge, as a potential marker for biological age, correlated well with another age-related biological marker, the plasma level of NAD+, pointing to the potential of this tool in future biology research on aging.

For the next step, we selected five common metabolic diseases for further evaluation based on our AI model. Our study showed that our AI model can accurately determine the subjects with these metabolic diseases (FIGS. 15A-15E). As these metabolic diseases have some common risk factors, including age and obesity (as determined by BMI), further analyses were conducted to evaluate the relative strength in their correlations with the diseases, which showed that our AI model determination was stronger than chronological age and BMI as an independent parameter in the identification of these metabolic diseases, with an exception of obesity, which perfect prediction was generated using BMI by definition (FIG. 15F). Obviously, for clinical utility, the next question will be whether a combination of the AI model and the other known clinical parameters will provide an even better algorithm. Further analysis showed that adding other clinical and laboratory parameters did not enhance the precision of the AI model assessment (FIG. 15F), further supporting the strong clinical value of our AI model based on the facial features/parameters.

To further determine the mechanism behind all these observations, we took on a major undertaking in evaluating plasma metabolites in these subjects. With a well-defined approach (FIG. 22), we applied multiple layers of screening and selection until the annotated metabolites were identified, which were then subjected to pathway enrichment analysis. The identification of the metabolic pathways that correlated with some of the facial features/parameters were extremely interesting and supported the original hypothesis of our study. For example, the levels of prostaglandin F2a, which was involved in arachidonic acid metabolism, are reported to be correlated with obesity and type 2 diabetes patients(Wang et al., 2018). The identification of the arachidonic acid metabolism linking to facial features in the region of eyelids was interesting (FIG. 17). In fact, the correlation of “inflammation” and puffy eyelids was also observed and recorded by the ancient Chinese medicine doctors. What's more, we identified vitamin related pathway of “Vitamin B6 metabolism” and the related vitamin B6 were associated with T2DM potentially because their role of antioxidant (Merigliano et al., 2018).

To our knowledge, this is the first attempt to link 3D facial features/parameters to biotypes and clinical diseases through AI deep learning. We have also identified metabolic pathways that are also tied to the facial features. It is important to note that in nearly all algorithms established by our AI model, both local regional features (including texture and morphology of the ROIs) and also global features were contributing to the algorithms, which can certainly not be able to be achieved with our regular human observations and our comprehension of such a complex nature of the multiple variables in the algorithm. Better facial data capture and AI deep learning are therefore, opening up a new dimension for the study of biology and clinical features in relation to the 3D facial features. We have chosen metabolic diseases as our first study but certainly, it is highly likely that this approach may also be very useful for some other diseases/biology as well. For this branch of new science based on 3D facial features/parameters, we would like to propose this discipline of science to be named “facial-omics”.

Finally, to ensure that this approach may eventually be able to be used clinically or even for individual use, we tested the general applicability of this approach using our AI model and adapted to a smartphone and using structured lighting with our AI program through an internet connection. The excellent AI determination in confirming a number of our previous observation in this prospective cohort further confirmed the potential role of this approach in the medical setting and even for personal use in the future. We are envisioning that one day, clinicians may use this approach as one of their clinical tools in the assessment of their patients and the public may also be able to use this tool as a general self-screening and monitoring with access to physicians for advice. It is also possible that this tool can assist the assessment of various environment factors that impact aging and evaluate therapeutic interventions that slow the aging process as determined by the measurement of the FaceAge (i.e. biological age).

This study has number of limitations. First, the overall size of the dataset, although already sizable (7,221), is still on the small side from a population-based perspective. More data and training for the AI will render this model more accurate and robust. Second, this study was conducted in Chinese and similar studies with different ethnic origins will be critical to further determine the general applicability of this approach. We contemplate that more novel findings will be identified with diverse ethnic populations. For example, detection of actinic keratosis in Caucasians, sickle-cell diseases among populations of African descent, and metabolic diseases that are more prevalent among different ethnic origins (such as Tay-Sachs disease). Third, there were still a lot to be learn from the other metabolites that we have detected, but their identity not yet determined, and we contemplate that there will be much more information to be extracted with this approach in the near future. Needless to say, further advancements of this approach will be facilitated by multi-country, multi-ethnic origin, and along multiple biology/disease segments in a collaborative fashion.

### Methods

### Image Datasets and Patient Characteristics

The 3D facial images were collected from the China Consortium of 3D Image Investigation cohort (CC-3DF), which consists of Han Chinese cohorts from China suboptimal health cohort study (COACS) and external cohort from Guangdong, China. Institutional Review Board (IRB)/Ethics Committee approvals were obtained in all locations and all participating subjects signed a consent form.

The China suboptimal health cohort study (COACS) is a community-based, prospective study, to investigate how suboptimal health status contributes to the incidence of NCD (non-communicable chronic diseases) in Chinese adults (Wang et al., 2016). This COACS study has two phases, a cross-sectional survey, followed by a longitudinal study. The participants were recruited from Tangshan city, which is a large, modem industrial city and adjoins two mega cities: Beijing and Tianjin. In phase I, all participants underwent clinical, laboratory and environmental exposure measurements aimed at identifying clinical, biological, environmental, and genetic factors associated with suboptimal health. In the second phase, a long-term yearly clinical follow-up have been performed until 2024, with the purpose of better understanding how suboptimal health, environmental and genetic risk factors contribute to the development of major chronic diseases. We have elected to use this cohort for our study because it has the balance of healthy subjects and those with metabolic diseases and medical records were relatively complete and previous electronic medical records were also available for assessment if needed.

The developmental cohort consisted of 7,221 patients from COACS with demographic information, life-style (smoking, alcohol intake) and clinical parameters from their electronic medical records were collected. If they consented to this study, they were selected whether they participate with the 3D face scanning, fasting blood draws and the use of the medical record data. 3D facial images were captured using 3dMDface camera systems (www.3dmd.com) beginning in the annual follow up study in 2019. Applying standard facial image acquisition protocols (Heike et al., 2010), participants were asked to close their mouths and hold their faces with a neutral expression for the capture of the digital facial stereophotogrammetry. 3D images in wavefront.obj file format with point clouds and corresponding texture images were used for further analysis.

Demographic and clinical data for all the study participants are summarized in Table 1. For each consenting subject, demographic and life-style (smoking, alcohol use) information, routine physical examination, and clinical laboratory were obtained. For patients who consented to additional fasting blood collection, plasma samples were collected for NAD+ and metabolomics analysis. For the NAD+ study, we included 806 healthy control subjects from the cohort. NAD+ levels of the blood plasma were measured using the cycling assay following a standard protocol (Li and Sauve, 2015). Because of experiment variations in sample processing, samples were processed in parallel. The NAD+ values from each subject were normalized and used to obtain the presented average values. For subjects consented for fasting blood draws and 3D face scanning, the ones with age below 20 or exceed 80 or with 3D facial image artifacts were excluded. The remaining 551 samples were used for further the metabolomics study (the processing and analysis of plasma samples are described below).

### Definitions and Criteria for Disease Diagnoses

The following criteria were used to define biometric parameters, lifestyle factors, and each disease category.

Smoking was defined as smoking on average of more than one pack (20 cigarettes)/day for at least one year.

Excessive alcohol use was defined as consuming on average of ≥60 ml per day for men and ≥30 ml per day for women.

Body mass index (BMI) was calculated as the body weight in kilograms divided by the square of body weight in meters.

Obesity was defined as BMI ≥30 kg/m².

Diabetes mellitus (Type II) was diagnosed by fasting blood glucose ≥7.0 mmol/L in a period of one year, or as an HbA1c value of 6% or more, and/or by a history of drug treatment for diabetes.

Metabolic Syndrome was defined as the presence of any three or more of the following: (1) Fasting blood glucose >6.1 mmol/L (110 mg/dl), or 2 h post-prandial glucose >7.8 mmol (140 mg/dl) or by a self-reported history of physician diagnosis of diabetes mellitus. (2) HDL cholesterol <0.9 mmol/L (35 mg/dl) in men, <1.0 mmol/L (40 mg/dl) in women and/or triglycerides >1.7 mmol/L, (150 mg/dl) (3) BMI >25 kg/m² (4) Systolic blood pressure ≥140 mmHg and/or diastolic blood pressure ≥90 mmHg, and/or self-reported current treatment for arterial hypertension.

Nonalcoholic Fatty Liver Disease (NAFLD) encompassed the spectrum of fatty liver disease confirmed by imaging or elastography and without significant alcohol consumption.

Hyperuricemia was defined as above uric acid level 420 μmol/L in men and above >360 μmol/L in women.

### Prospective Pilot Study at a “Point of Care” Setting Using a Smartphone

An AI model was initially developed using 3D facial images taken by a 3dMDface camera system. With the rapid advancement of 3D smartphone systems, we also explored the potential of developing a “point-of-care” system by using some advanced camera features in smartphones to explore the general applicability of our AI model. For this prospective independent cohort in subjects enrolled in Guangzhou, 3D images in a wavefront.obj file format were obtained using smartphones with a structured light module. A total of 432 patients were included in the cohort (Supplemental Table 1). This external cohort included subjects for the analysis of four metabolic diseases (including hyperuricemia, T2DM, metabolic syndrome, obesity) and normal controls. Demographic and clinical data for the pilot study participants are summarized in Supplemental Table 1. We did not have sufficient number of subjects with NAFLD in this cohort.

### 3D Facial Image Preprocessing

3D images in a wavefront.obj file format was utilized, which generated a dense 3D points cloud representing the surface geometry of the face from multiple 2D images with overlapping fields of view. Three steps of pre-processing were involved, including landmark detection, alignment, and multi-view projection, to pre-process the 3D images.

### Landmark Detection

To localize and represent salient regions of the face, we identified a total of 80 facial landmarks including of 65 landmarks generated by deep learning models and 15 auxiliary landmarks generated based on known landmarks. Deep Convolutional Neural Network (DCNN) based method was used to detect landmarks on 3D facial images, of which 65 landmarks were used for in the study for further analysis. (Fagertun et al., 2014). The input of 3D facial image was randomly projected into multi-views (100 times) (Paulsen et al., 2019) and further fed to the trained DCNN models to generate 2D heatmaps of landmarks. Finally, a Least Squares (LSQ) fitting method combined with Random Sample Consensus (RANSAC) (Fishier and Bolles, 1980) were utilized to integrate the 2D heatmap results generated from different views to generate the accurate 3D facial landmarks. In addition, to cover ROIs such forehead, cheekbone and nasal bone, 15 auxiliary landmarks were generated by a linear combination of known landmarks.

### Alignment

All 3D facial images were aligned with a template facial image as reference (Claes et al., 2018). Based on the landmarks of the template and the 3D facial images, rigid registration method was performed to generate similarity transformation matrices. Then the transformation matrices were applied on the input 3D facial images to establish rough alignments. Spatially dense alignments were established by corresponding point matching between the template and the 3D facial image and a standardized 3D facial image was established.

### Multiview Projection

First, the frontal view of 3D facial surface was obtained by adjusting the horizontal direction according to the corners of eyes and adjusting the vertical direction according to the connection vector between the center of the corners of two eyes and the center of the corners of mouth. To obtain information of the all-round information of the 3D face, the 3D face was rotated and projected in 13 views of directions, which included the frontal view, 3 views for every 10 degrees from up, down, left and right, respectively.

### Extraction of Facial-Omics Information

Facial-omics was defined as performing extraction of high throughput information as quantitative descriptors from the 3D faces. A region of interest (ROI) based method was used to extract facial-omics. The ROIs of facial images are the cropped surface areas based on preexisting anatomical knowledge. From the segmented ROIs, the local feature information, named facial-omics, which includes the morphology features and the texture features of the facial images were extracted.

For ROI segmentation of the facial images, a contour optimization approach was employed (Clements and Zhang, 2006; Cohen, 2006) to automatically generate 20 ROIs without an overlap. The landmarks were used to define the segmented surface areas. The 20 ROIs are illustrated in FIG. 19B, which include surface areas of forehead, glabella, eye upper left, eye upper right, eye left, eye right, eye corner left, eye corner right, eye lower left, eye lower right, top nose, nose side left, nose side right, cheek left, cheek right, mouse, temple left, temple right, philtrum, chin. A three-dimensional graph was employed to represent the landmarks and surfaces of a 3D face, where each ROI was regarded as a sub-surface of the face graph surrounded by a closed path. A path indicates the curved line passed through the neighbour landmarks. For each ROI, a set of minimal paths connecting pairs of facial landmarks formed a closed contour. Thus, the k-th ROI could be represented as pₖ=[vₖ₁, vₖ₂, . . . , vₖₙ, vₖᵢ], where vₖᵢ is the i-th landmark in pₖ. FIG. 19 shows all defined ROIs with corresponding landmarks and paths.

After the 3D facial images were segmented, the Principal Component Analysis (PCA) was applied to extract major features of morphology variations for each facial ROI. The PCA analysis is a commonly used approach for dimensionality reduction and could eliminate some noisy and meaningless shape variations that result from various sources of error (Claes et al., 2018). The linear combination of Principal Components (PCs) was extracted from the given segment as the morphology features. Given a defined ROIR, it was represented with a morphology-vector M=(x₁, y₁, z₁, . . . , xₙ, yₙ, zₙ)∈ [symbol] ³ⁿ, where n is the number of vertices containing x,y,z-coordinates. PCA was then performed to all morphology-vectors of corresponding ROI from training data, so that morphology variations of R could be obtained using a linear combination of k reduced dimensions of PCs.

In addition to morphology features, texture features represented the local brightness, structure patterns, or the spatially repetitive structure of surfaces such as local variations of scale, orientation, or other geometries, which were considered to be important visual patterns of facial components (Kaesemodel Pontes et al., 2015). First, multi-views of facial images were converted to grayscale. A total of 10 typical texture features were extracted for each ROI, including kurtosis, skewness, standard deviation, contrast, correlation, uniformity, directionality, homogeneity, coarseness, and directionality. Among them, kurtosis, skewness, standard deviation are first-order statistical texture features. The kurtosis was extracted to describe the sharpness of the histogram and the skewness was defined as the degree of asymmetry around the mean value. Contrast, correlation, uniformity, directionality and homogeneity are second-order statistical texture features (Lambin et al., 2012). Of these, contrast, uniformity, directionality and homogeneity also belong to the set of visual features of texture proposed by Tumura et al. (Tamura et al., 1978). The other two visual texture features of coarseness and directionality were also quantified as facial-omics. Coarseness relates to distances of dominant spatial variations of grey levels, that is, implicitly, to the size of the primitive elements (texels) forming the texture. Degree of directionality measures the frequency distribution of oriented local edges against their directional angles. For the second-order statistical texture features, gray-level co-occurrence matrix (GLCM) was employed to analyze the spatial distribution of image texture through different spatial positions and angles to represent visual texture characteristics (Zhao et al., 2014).

### Deep Learning Model for Global Feature Extraction

A deep convolutional neural network was pretrained for global feature extraction using the publicly dataset IMDB-WIKI (Rothe et al., 2015), which is a large scale dataset of 523,051 2D facial images with age and gender labels. ResNet-50 (He et al., 2016) was used as backbone of our network. ResNet-50 is a five-stage network with a convolution and four identity blocks, which utilizes skip connections to overcome the degradation problem of deep learning models. We modified the last global averaging layer of the network to 512 nodes for the 512-dimensional global feature extraction. Considering that the age values are discrete numeric numbers in a [0,100] range in IMDB-WIKI and our dataset, a fully connected layer with 101 nodes was appended when pretraining on IMDB-WIKI. After the pretraining process, the fully connected layer was removed. Finally, the network with retained structures and parameters were used as our global feature extractor. Given a face image, the face image was resized to 512×512: and fed into the extractor to obtain 512-dimensional feature vectors. To improve generalization of deep learning models, we applied data augmentations: oversampling (Masko and Hensman, 2015) brightness, contrast, saturation, and rotation.

### Joint Model: Combining Global Features and Facial-Omics

A joint model was constructed for clinical parameter prediction and metabolic disease classification. The joint model was a multiple layer perceptron, integrating global features and local features of the same subject as an input. A total of 489 local features were concatenated with 512 global features of the same subject. Then two fully connected layers with ReLU activation function were used for different tasks.

Three separate models were trained with different last fully connected layers for three tasks, including prediction with clinical parameters (except age), assessment of age, and multiple binary classification of metabolic diseases. We used the Mean-Square Error (MSE) loss as the objective function for the prediction of clinical parameters except age and the Binary Cross Entropy (BCE) loss for multiple binary classification. For age assessment, we used a separate model to achieve better prediction performance by using ordinal Regression (Pan et al., 2018). We considered age assessment as a classification task and used the expected value over the softmax output probabilities. Cross entropy loss with a regularization of mean-variance loss was employed for age assessment. Mean-variance loss consists of two penalization items for a concentrated distribution, λ₁(m−y)² and λ₂v, where (m−y) is the difference between the mean m of the estimated age distribution and the ground-truth age y, and v is the variance of the estimated age distribution. We set the weights α₁=0.4 and α₂=0.05 in our experiments, which were previously shown to work well for age correlation/prediction(Pan et al., 2018).

The models were implemented using PyTorch (Paszke et al., 2019), and optimized by the Adam algorithm (Kingma and Ba, 2014) with a learning rate of 0.001 and a weight decay of 10⁻⁴. The training was conducted over 50 epochs across the dataset with a batch size of 32 samples. The model training and evaluation was based on 10-fold cross-validation. Thus, all samples were split into mutually exclusive sets for training and validation (90%) and testing (10%). This process was repeated 10 times, yielding a total of 10 mutually exclusive test sets that were collectively exhaustive.

Prediction of Clinical Parameters and Metabolic Diseases with 3D Facial Images

The correlations between the actual clinical parameters and projected clinical parameters by 3D facial images was assessed. Our model was trained using MSE loss to regress clinical parameters (including height, weight, BMI, blood uric acid level). Pearson's correlation test was further performed between actual values and projected values. The resultant correlations were regarded as significant when P-values were 0.001.

To study the correlation between biological age and life-style, our model was trained for age projection on 5,463 normal subjects without the habit of smoking and excessive alcohol use for 10-fold cross-validation. The projected results were integrated on each-fold test dataset to compute the correlation of chronological age and FaceAge (FIG. 15A), and then the trained model was applied to project FaceAge for the 967 subjects with smoking habit and 1,406 subjects with excessive drinking habit. The cross validation was performed at the patient level guided by individual ID to ensure that all images from the same patient were allocate to at most one subset per cross. The two-side P-values were computed by the Student's t-test for non-smoking vs smoking and non-drinking vs drinking with AgeDiff (FIGS. 15B and 15C), which was defined as predicted FaceAge minus the chronological age.

The correlation between age (chorological age and biological FaceAge) and plasma NAD+ levels form healthy controls (n=806) was also studied. Multivariate regression analysis was applied on the effect of age on blood NAD+ levels, adjusting for gender and BMI as the following: Adjusted NAD⁺=NAD⁺−a*Gender−b*BMI, where a and b are the coefficients of the fitting linear model. After predicting age for healthy subjects, we used Fisher's r-to-z transformation and one-tail t test (Fisher, 1921) to get whether there was statistical significance between FaceAge-NAD+ distribution and chronological age-NAD+ distribution. The multivariate analysis was implemented by statsmodels, a Python package (Seabold and Perktold, 2010) Two multivariate regression models were built, on chronological age or FaceAge, respectively. The resultant coefficients were regarded as significant when P-values were <0.001.

The detection of metabolic disease or lifestyle task was treated as multiple binary classification. We used 3D facial images from 3dMDface camera systems to predict metabolic diseases or lifestyles with the joint model that combine the global features and facial-omics. Metabolic diseases, including obesity, T2DM, metabolic syndrome, nonalcoholic fatty liver disease (NAFLD), and hyperuricemia, were included for prediction analysis in the study. The AUC was calculated using the output probability of the AI model and the actual label on the test set. BMI and age have previously been shown to be risk factors of metabolic diseases and were predictable using facial images. Therefore, to ensure that the AI model were not identifying metabolic diseases via BMI and age, we first developed logistic regression models using the clinical metadata of age, BMI separately. We also explored the impact of combing of all three elements (age, BMI and the AI model) as input of the logistic regression model for disease prediction.

To visualize of the biomarkers or metabolic diseases on the facial ROI map, we used the gradient-based method to generate ROI saliency maps for interpretation of our models (Rs et al., 2016). First, the gradients of all facial omics at feature level were derived via the loss from predicted labels and supervised labels. Then we aggregated gradients of the features (including morphology features and texture features) for each ROI to obtain the saliency into ROI-level. We obtained average ROIs saliency across all samples and normalized them into to [0,1]. The saliency map was visualized on the ROIs on the 3D face with a value larger than 0.3.

### Metabolomics Analysis

### Sample Preparation and Data Acquisition

We performed metabolomics profiling of 551 subjects (91 individuals with T2DM and 460 healthy controls) consented in the cohort. Human plasma samples of the 551 subjects were prepared according to a previous report (Contrepois et al., 2015). Plasma samples were thawed on ice, prepared, and analyzed in a randomized order. Plasma was treated with four volumes of a acetone:acetonitrile:methanol (1:1:1, v/v) solvent mixture and incubated for 2 h at 20° C. to allow protein precipitation, then it was centrifuged at 10,000 rpm for 10 min at 4° C. and evaporated to dryness. The residues were reconstituted with 50% methanol before analysis.

### UPLC-Q-TOF/MS Analysis

Liquid chromatographic (LC) separation for processed plasma was achieved on a ACQUITY UPLC BEH C18 column (2.1×100 mm 1.7 μm) using an ACQUITY Ultra Performance LC (Waters Corporation, Milford, Massachusetts, USA). Mobile phase A and B were 0.06% acetic acid-containing water and 0.06% acetic acid-containing methanol, respectively, and the gradient was set as follows: 0-10 min, 1%-80% B. The oven temperature was set to 60° C., the injection volume was 5 μL and the flow rate was 0.4 mL/min.

Mass spectrometry was performed on a SYNAPT G2 Quadrupole-Time of Flight system (Waters Corporation, Milford, Massachusetts, USA). During analysis of the samples, 1 quality control sample was run after every 20 injections. The Q-TOF was operated in positive and negative full scan mode. The data were recorded between the range of 50-1,000 m/z. The MS parameters were as follow: gas temperature 325° C., drying gas flow 91/min, nebulizer 45 psig, fragmentor 125 V, capillary voltage 3,500 V.

Metabolic Biomarkers Associated with Metabolic Disease and Facial-Omics Features

LC-MS/MS data alignment, detection of the peaks, adduct deconvolution, normalization, and identification was performed using Progenesis QI (Waters, Nonlinear Dynamics, UK) software (version 2.4). The mass tolerance in MS and MS' was 5 ppm and 10 ppm, respectively. For the 551 subjects, to avoid the impact of obesity as a confounding variable, we excluded the obese individuals. The remaining samples consisted of 533 subjects (83 individuals with T2DM and 450 healthy controls) in the cohort. Using a high-throughput metabolomics approach, 8,037 metabolite peaks were detected in patients' plasma samples using both negative ionization and positive ionization methods. In total, 3,560 metabolite features were selected for further analysis after passing the initial quality control and removing peaks with missing values. For the 3,560 metabolite features, we performed a parallel analysis to map the metabolite peaks to facial-omics and differentiate metabolic disease versus control groups. The metabolites that both associated with facial omics and type 2 diabetes were further used for pathway analysis.

First, all 3,560 metabolite features were mapped onto facial-omics at an ROI-level using a canonical correlation analysis (CCA). CCA is a multivariate statistical analysis of correlation between two vectors of random variables. For each facial ROI, CCA extracted the linear combination of morphology or texture features that had maximal correlation with metabolic biomarkers. We calculated Pearson's correlation by using metabolites abundance and combined value of morphology/texture feature at ROI-level. P-values adjusted using false discovery rate (FDR)<0.01 were tested for significance.

In parallel, we performed nonparametric univariate method (Wilcoxon Rank Sum test) with Fold Change analysis to identify metabolic biomarkers with significant differential abundance in type 2 diabetes. Multivariate statistical analyses (orthogonal partial least-squares discrimination analysis, OPLS-DA) were applied to discriminate Type 2 diabetes individuals from controls using an R package named “ropls” (Thevenot et al., 2015). OPLS-DA is a supervised analysis method, where the metabolites were set as the projecting factors and metabolic disease was set as the response. 1,101 metabolite features driving the difference in the metabolic profiles between T2DM individuals and controls were identified based on degrees of importance in the projection (VIP) score (VIP score >1) from the OPLS-DA model. At the same time, with Wilcoxon test and Fold Change analysis to identify T2DM-related metabolic peaks from the initial 3,560 candidates, which revealed 394 significant metabolite peaks (blue and red colored dots) on a volcano plot (Fold Change>1.5 in any direction and FDR<0.05). Combining the above univariate and OPLS-DA approaches, a total of 354 metabolite peaks were finally identified as differential metabolites of T2DM. We further obtained 328 metabolite peaks both correlated with facial omics and type 2 diabetes.

Metabolite mapping and annotations were performed using the Human Metabolome Database (HMDB) and the METLIN Metabolite and Chemical Entity Database (http://metlin.scripps.edu) for MS and MS/MS-based metabolite identification. Metabolic biomarkers which were significantly correlated to both metabolic disease and facial-omics were search and matched to known metabolites and used for a functional enrichment analysis. For the annotated metabolites, we performed the regularized partial correlation network analysis using “qgraph” package in R. The tuning parameter gamma(γ), which controls the complexity of the network, was set to 0.5 as suggested (Epskamp and Fried, 2018). In the network, node represents a compound, and each edge represents the strength of partial correlation between two nodes after conditioning on all other variables in the datasets. Pathway enrichment analysis of these metabolic biomarkers were further performed using the Kyoto Encyclopedia of Genes and Genomes (KEGG; Homo sapiens) pathway library with a MetaboAnalyst v.4.0 software package (Chong et al., 2018). The statistical significance was evaluated by global testing which was provided in MetaboAnalystR, a tool designed for metabolomics analysis to gain biological insights into the functional roles of pre-defined subsets of metabolites (Goeman et al., 2004).

### Quantification and Statistical Analysis

The deep learning model performance for age projection was evaluated with three evaluation metrics including Mean Absolute Error (MAE), R-square (R2) and Pearson Correlation Coefficient (PCC). The MAE is the measure of errors between predicted age and chronological age. The R2 is a statistical measure that represents the proportion of the variance for predicted age explained by chronological age in our deep learning model. The PCC was used to measure the correlation between two variables. It has a value between +1 and −1, where +1 and −1 is total complete linear correlation and 0 is no linear correlation. The significance of correlation between two distributions were computed using bootstrapping (Efron, 1992) approach with resampling of 1000 times. Receiver operating characteristics (ROC) and Area under the Curve of ROC (AUC-ROC) were employed to assess model performance for each classification task. The ROC curves were plotted by using the true positive rate (sensitivity) versus the false-positive rate (1−specificity). Python scikit-learn library was used for data analysis and plotted graphs was performed with the Python matplotlib and seaborn libraries.

### FIGURE LEGEND

FIG. 13. (A) 3D facial image representation. A 3D facial image was segmented into regions of interests (ROIs) based on 80 landmarks. From the corresponding facial ROIs, the features were extracted to train the AI model. The AI model consisted of two modules for facial representation: the local feature extraction module and the global feature extraction module. The local feature information (named facial-omics) was consisted of the morphology features and the texture features of 3D face (see Methods for more details). (B) We used a joint model which combined facial-omics and global features for prediction of metabolic disease and clinical parameters. A prospective pilot study was also conducted using 3D images taken from a smartphone to test our AI performance for clinical applications. (C) Workflow chart for the metabonomic analysis. Metabolites were identified linking the facial omics (the quantitative features of 3D face, see Methods for details) and metabolic diseases. In this approach, metabolite features were mapped onto facial omics features to identify facial-associated metabolic biomarkers. In parallel, metabolites differentially present in metabolic diseases were identified. The shared metabolites between facial omics and metabolic disease were subject for a pathway enrichment analysis (FIG. 19).

FIG. 14. (A) Correlation between chronological age and predicted age: Pearson correlation coefficient (PCC)=0.93, mean absolute error (MAE)=2.79, coefficient of determination (R2)=0.96. Predicted age was calculated using the 3D facial images. (B and C) The AgeDiff analysis of lifestyle factor modifications on predicted biological age (FaceAge). AgeDiff measured the difference of predicted biological age and chronological age. Box plots showed median, upper quartile and lower quartile (by the box) and the upper adjacent and lower adjacent values (by the whiskers). (B) Increased biological age of smokers comparing to non-smokers. (C) Increased biological age of alcohol users comparing to non-alcohol users. (D) Decline of NAD+ levels with an increased age. The blue dots and line denoted the predicted FaceAge (biological age). The orange dots and line denoted the actual chronological age. (E-G) Linear regression analysis of the actual and the predicted clinical parameters, including (E) body weight, (F) height, (G) BMI. See Methods for details.

FIG. 15. AUC curves of the binary classification with 95% CIs were calculated using 1,000 bootstrap samples. (A) Binary classification of obesity and control groups: AUC-ROC=0.907 (95% CI: 0.894, 0.920). (B) Binary classification of T2DMs and control groups: AUC-ROC=0.842 (95% CI: 0.827-0.856). (C) Binary classification of metabolic syndrome and control groups: AUC-ROC=0.861 (95% CI: 0.846-0.874). (D) Binary classification of NAFLD and control groups: AUC-ROC=0.865 (95% CI: 0.851-0.878). (E) Binary classification of hyperuricemia and control groups: AUC-ROC=0.831 (95% CI: 0.819-0.842). (F) Performance of identification of metabolic diseases with contribution from age, BMI, face, and the combination of the three factors. The predictions of common metabolic diseases included obesity, diabetes, metabolic syndrome, NAFLD, and hyperuricemia. “FaceAI” denoted the AI model based on 3D facial images. NAFLD, Nonalcoholic Fatty Liver Disease. Obesity was defined as BMI >30 kg/m².

FIG. 16. (A) Heatmap of correlations between metabolite features and facial-omics at an ROI-level. The left of the heatmap denoted the morphology features and the right of the heatmap denoted the texture features. A canonical correlation analysis (CCA) was used to extract the linear combination of morphology/texture features from a ROI segment. 1,897 metabolite features showed significantly correlations with features of ROIs segments (FDR <0.01). (B) A volcano plot showing differential metabolite features enriched in T2DM patients. In this volcano plot, blue and red dots denoted metabolite features showing significant changes between T2DM group versus controls (FDR<0.05, Fold Change>1.5 in any direction). The scatter-plot represented significance (p-value, −Log 10) versus fold-change (Log 2) on the y and x axes, respectively. (C) Pathway enrichment analysis of 20 annotated metabolites using the Kyoto Encyclopedia of Genes and Genomes (KEGG) pathway library.

FIG. 17. The 20 shared metabolites between T2DM and the facial-omics were associated with distinct ROIs. Inside the circle: The 20 annotated metabolites that both associated with diabetes and facial-omics. Outside the circle: Display of RIOs on the facial map corresponding to each of 20 metabolites. The associated ROI segments were in green color.

FIG. 18. (A) Correlation of chronological age and predicted age: PCC=0.94, MAE=3.92, R2=0.88. Predicted age was calculated using the 3D facial images. (B-C) The AgeDiff analysis in indicated groups relating to lifestyle factors. (B) Increased biological age in smokers comparing to non-smokers. (C) Increased biological age of alcohol users comparing to non-alcohol users. (D-G) Performance of the AI model on classification of metabolic diseases shown as AUC curves of the binary classification. (D) Obesity, AUC-ROC=0.898 (95% CI: 0.845-0.942). (E) Type 2 diabetes, AUC-ROC=0.805 (95% CI: 0.727-0.875). (F) Metabolic syndrome, AUC-ROC=0.820 (95% CI: 0.708-0.917). (G) Hyperuricemia, AUC-ROC=0.814 (95% CI: 0.737-0.888). (H-I) Linear regression analyses of the actual and the predicted clinical parameters, including (H) body weight, (I) height.

FIG. 19. The ROIs of facial images represented the cropped skin areas based on anatomical information. ROI, region of interest. (A) Multiview projections of a 3D face. To obtain information of an all-round 3D face, the 3D face was rotated and projected in 13 directions of views. Upper panel: projected faces by an azimuth viewing from −30 to +30 degrees relative to a frontal view. Lower panel: projected faces from progressive chin-down to chin-up views from −30 to +30 degrees relative to the frontal view. (B) An example of a composite facial image with an ROI map based on the landmarks. Images from 50 individuals were used to create this composite photograph. Blue numbers indicated the landmark indices. The blue line connecting the neighbor landmarks indicated the path in FIG. 19C. The red numbers indicated the ROI indices which was surrounded by a closed path. (C) The segments of ROIs of a 3D face. Each ROI was a region of the facial surface surrounded by a circular path defined by landmarks. A path denoted the curved line passed through the adjacent landmarks. (D) Illustration of 20 segmented ROIs on a 3D face.

FIG. 20. (A and B) Correlation of predicted FaceAge and chronological age. Predicted FaceAge (biological age) was calculated using 3D facial images of (A) male subjects or (B) female subjects. (C and D) Correlation of NAD+ levels with age. The blue dots and line denoted the predicted FaceAge (biological age). The orange dots and line denoted the actual chronological age. (C) plot generated with Male subjects. (D) plot generated with female subjects. (E-G) Performance of the AI model on identification of gender and lifestyle factors (smoking and alcohol use). Shown as AUC curves of a binary classification of the AI model. 95% CIs were calculated using 1,000 bootstrap samples. (E) Gender: AUC-ROC=0.998 (95% CI: 0.995-1.000). (F) Smoking: AUC-ROC=0.863 (95% CI: 0.845-0.879). (G) Alcohol use: AUC-ROC=0.834 (95% CI: 0.821-0.848).

FIG. 21. (A-I) The “green color areas” highlighted the skin areas relevant to the model prediction. (A) Age, (B) Gender, (C) Smoking, (D) Alcohol use, (E) Obesity, (F) T2DM, (G) Metabolic syndrome, (H) NAFLD, (I) Hyperuricemia.

FIG. 22. Metabolite features (3650) from plasma samples was obtained after passing the initial quality control and filtering out missing values. 1,897 metabolite peaks were identified to be associated with facial-omics by the canonical correlation analysis (CCA) (FDR<0.01). In parallel, 354 metabolic features differed in abundance between T2DM and control groups were identified using an orthogonal partial least-squares discrimination analysis (OPLS-DA) and a Wilcoxon test (FDR<0.05, Fold change >1.5 in any direction). 328 metabolite features were further identified as the overlapping ones that both correlated with the T2DM and facial-omics. The 328 metabolites were searched and annotated for known metabolites using the Human Metabolome Database (HMDB) and METLIN databases (http://metlin.scripps.edu). 20 known metabolites were identified and subject to a pathway enrichment analysis using Kyoto Encyclopedia of Genes and Genomes (KEGG) database.

FIG. 23. (A) An OPLS-DA score plot showing clustering and separation of T2DM versus control groups. OPLS-DA (orthogonal partial least squares discriminant analysis) was used to perform multivariate modeling of metabolic disease. Models with one predictive (p1) and one to three orthogonal components (o1-o3) were built with the metabolites from the initial 3,560 metabolites. Metabolites were set as predictors and metabolic diseases as the response. The ellipse circles represented 95% of the multivariate normal distributions with the samples covariances for each class. (B-C) The correlation of two representative metabolites, (B) Oxoglutaric acid and (C) L-Cystine, with individual features of facial-omics. The two metabolites were significantly enriched in the KEGG pathways. Pearson's correlation test was performed. The X-axis denoted the 20 ROI segments of the 3D face. The Y-axis denoted the quantitative facial-omics features in each segmented skin areas of the ROIs, including skew, kurtosis, correlation, homogeneity, coarseness and directionality and morphology. (D) Correlation network of compounds differentially present in T2DM versus controls. Here, each node represented a compound, and each edge represents the strength of the correlation between two compounds after conditioning on all other compounds in the datasets.

### Tables

TABLE 1
Baseline demographics and data characteristics of the study cohort.

| | | Developmental dataset |
|---|---|---|
| Characteristics | | (n = 7,221) |
| Number of images | | 7,221 |
| Age, years | | 46.93 ± 13.67 |
| Gender | Male | 3,493 (48.4%) |
| | Female | 3,728 (51.6%) |
| Life-style | Smoking^{a} | 967 |
| factors | Alcohol use^{b} | 1406 |
| BMI, kg/m² | | 24.50 ± 3.51 |
| Height, cm | | 167.23 ± 8.46 |
| Weight, kg | | 69.18 ± 13.02 |
| Metabolic | Obesity^{c} | 442 |
| diseases | Diabetes^{d} | 699 |
| | Metabolic syndrome^{e} | 1063 |
| | NAFLD^{f} | 762 |
| | Hyperuricemia^{g} | 1,329 |

^{a}Smoking was defined as smoking for an average of one pack (20 cigarettes)/day for at least one year.
^{b}Excessive alcohol use was defined as average of ≥ 60 ml per day for men and ≥ 30 ml per day for women.
^{c}Obesity was defined as BMI ≥ 30 kg/m².
^{d}Diabetes mellitus (Type II) was diagnosed by fasting blood glucose ≥ 7.0 mmol/L in a period of one year, or as an HbA1c value of 6% or more, and/or by a history of drug treatment for diabetes.
^{e}Metabolic syndrome was defined as the presence of any three or more the following: (1) Fasting blood glucose > 6.1 mmol/L (110 mg/dl), or 2 h post-prandial glucose > 7.8 mmol (140 mg/dl) or by a self-reported history of physician diagnosis of diabetes mellitus. (2) HDL cholesterol < 0.9 mmol/L (35 mg/dl) in men, <1.0 mmol/L (40 mg/dl) in women and/or triglycerides > 1.7 mmol/L (150 mg/dl). (3) BMI > 25 kg/m². (4) Systolic blood pressure ≥ 140 mmHg and/or diastolic blood pressure ≥ 90 mmHg, and/or self-reported current treatment for arterial hypertension.
^{f}Nonalcoholic Fatty Liver Disease (NAFLD) encompasses the spectrum of fatty liver disease confirmed by imaging or elastography without significant alcohol consumption.
^{g}Hyperuricemia was defined as uric acid level above 420 μmol/L in men and above 360 μmol/L in women.

SUPPLEMENTAL TABLE 1
Clinical parameters and data
characteristics of the pilot study.

| | | Pilot study (using smartphone) |
|---|---|---|
| Characteristics | | (n = 432) |
| Number of images | | 432 |
| Age, years | | 48.63 ± 17.30 |
| Gender | Male | 229 (53.0%) |
| | Female | 203 (47.0%) |
| Life-style | Smoking | 101 |
| factors | Alcohol use | 122 |
| BMI, kg/m² | | 22.57 ± 3.76 |
| Height, cm | | 163.63 ± 8.10 |
| Weight, kg | | 59.72 ± 11.95 |
| Metabolic | Obesity | 27 |
| diseases | Diabetes | 49 |
| | Metabolic syndrome | 54 |
| | NAFLD | N/A |
| | Hyperuricemia | 77 |

The above description of the disclosed embodiments is provided to enable any person skilled in the art to make or use the invention. Various modifications to these embodiments will be readily apparent to those skilled in the art, and the general principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

Combinations, described herein, such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, and any such combination may contain one or more members of its constituents A, B, and/or C. For example, a combination of A and B may comprise one A and multiple B's, multiple A's and one B, or multiple A's and multiple B's.

1. A method comprising using at least one hardware processor to:
train an artificial intelligence to predict at least one clinical parameter or medical condition from facial images by training a first convolutional neural network to detect facial landmarks in each facial image,
training a second convolutional neural network to predict one or more global features from each facial image,
generating a facial-omics model to predict one or more local features from each facial image, and
training a classification model to predict the at least one clinical parameter or medical condition based on the one or more global features and the one or more local features; and
operating the trained artificial intelligence by, for each of a plurality of facial images, receiving the facial image,
applying the first convolutional neural network to identify the plurality of facial landmarks in the facial image,
aligning the facial image to a template based on the identified plurality of facial landmarks,
applying the second convolutional neural network to the aligned facial image to predict the one or more global features,
applying the facial-omics model to the aligned facial image to predict the one or more local features, and
applying the classification model to the one or more global features and the one or more local features to generate a prediction of the at least one clinical parameter or medical condition for the facial image.

2. The method of claim 1, wherein receiving the facial image comprises receiving the facial image from a mobile device, which captured the facial image, over at least one network.

3. The method of claim 1, wherein one or both of the first convolutional neural network and the second convolutional neural network comprise a deep convolutional neural network.

4. The method of claim 3, wherein the second convolutional neural network comprises a ResNet-50 in which a last global averaging layer is modified to produce an N-dimensional vector of global features, wherein N is greater than one hundred, such that the one or more global features comprise more than one-hundred global features.

5. The method of claim 1, wherein aligning the facial image to a template based on the identified plurality of facial landmarks comprises computing a transformation that moves each of the identified plurality of facial landmarks in the facial image to a corresponding position of that facial landmark in the template.

6. The method of claim 1, wherein each received facial image is a three-dimensional facial image, and wherein applying the second convolutional neural network to the aligned facial image to predict the one or more global features comprises:
projecting the aligned three-dimensional facial image into a plurality of two-dimensional directional views, wherein each of the plurality of two-dimensional directional views is a view of the three-dimensional facial image from a different angle than the other plurality of two-dimensional directional views; and
applying the second convolutional neural network to the plurality of two-dimensional directional views to predict the one or more global features.

7. The method of claim 6, wherein the plurality of two-dimensional directional views comprises a frontal view of a face in the three-dimensional facial image, one or more views of the face rotated in a leftward direction relative to the frontal view, one or more views of the face rotated in a rightward direction relative to the frontal view, one or more views of the face rotated in an upward direction relative to the frontal view, and one or more views of the face rotated in a downward direction relative to the frontal view.

8. The method of claim 7, wherein the one or more views of the face rotated in the leftward direction, the one or more views of the face rotated in the rightward direction, the one or more views of the face rotated in the upward direction, and the one or more views of the face rotated in the downward direction all comprise a plurality of views at fixed intervals of rotation.

9. The method of claim 8, wherein each plurality of views comprises at least three views.

10. The method of claim 1, wherein the facial image is a three-dimensional facial image, and wherein applying the facial-omics model to the aligned facial image to predict the one or more local features comprises:
segmenting the three-dimensional facial image into a plurality of regions of interest; and
applying the facial-omics model to the plurality of regions of interest to extract local features from each of the plurality of regions of interest.

11. The method of claim 10, wherein the plurality of regions of interest are non-overlapping, and wherein the plurality of regions of interest comprises a corner of right eye, right side of nose, upper right eye, right eye, lower right eye, chin, glabella, forehead, right cheek, philtrum, right temple, nose, mouth, corner of left eye, left side of nose, upper left eye, left eye, lower left eye, left cheek, and left temple.

12. The method of claim 10, wherein segmenting the three-dimensional facial images into a plurality of regions of interest comprises:
representing the three-dimensional facial image as a face graph; and
connecting subsets of the identified plurality of facial landmarks in the face graph into cycles representing the plurality of regions of interest.

13. The method of claim 10, wherein the facial-omics model comprises principal component analysis.

14. The method of claim 10, wherein the local features comprise one or both of one or more morphological features or one or more textural features.

15. The method of claim 14, wherein the local features comprise a plurality of textural features, and wherein the plurality of textural features comprises kurtosis, skewness, standard deviation, contrast, correlation, uniformity, directionality, homogeneity, coarseness, and directionality.

16. The method of claim 1, wherein the at least one clinical parameter or medical condition comprises one or more of the following clinical parameters: age, weight, height, body mass index, smoking use, alcohol consumption, alanine aminotransferase, uric acid, hemoglobin concentrations, glutamyltransferase, hematocrit, and red blood cell volume.

17. The method of claim 1, wherein the at least one clinical parameter or medical condition comprises one or more of the following medical conditions: obesity, diabetes, metabolic syndrome, hyperuricemia, nonalcoholic fatty liver disease, and anemia.

18. The method of claim 1, wherein the classification model comprises a multilayer perceptron that outputs a vector of probabilities for a plurality of classifications representing the at least one clinical parameter or medical condition.

19. The method of claim 1, wherein the classification model comprises a first model for predicting one or more clinical parameters other than age, a second model for predicting age, and a third model for predicting one or more medical conditions.

20.-21. (canceled)
